(19) United States
(12) Patent Application Publication
(10) Pub. No.: US 2025/0333687 A1
(43) Pub. Date: Oct. 30, 2025

(54) Controlling Yeast Populations with Inter-Domain Genetic Modification via Conjugation Mediated Genetic Transfer

(71) Applicant: Wisconsin Alumni Research Foundation, Madison, WI (US)

(72) Inventors: Megan McClean, Fitchburg, WI (US); Kevin Stindt, Madison, WI (US)

(73) Assignee: Wisconsin Alumni Research Foundation, Madison, WI (US)

(21) Appl. No.: 19/041,404

(22) Filed: Jan. 30, 2025

Related U.S. Application Data
(60) Provisional application No. 63/548,971, filed on Feb. 2, 2024.

(51) Int. Cl. C12N 1/16; A61K 35/74; A61P 31/10; C12N 1/20; C12N 9/22; C12N 15/11; C12N 15/70; C12R 1/19

(52) U.S. Cl. CPC C12N 1/16; A61K 35/74; A61P 31/10; C12N 1/20; C12N 9/226; C12N 15/111; C12N 15/70; C12N 2310/20; C12N 2800/101; C12R 2001/19

(57) ABSTRACT

Described herein is a method of fungal population modification via inter-domain conjugation, including co-culturing a bacterial population and the fungal population under growth conditions for the bacteria, and maintaining growth of the bacterial population during the co-culturing by controlling an essential nutrient for growth of the bacterial population. A bacterial plasmid is transferred to at least a portion of the fungal population to provide the inter-domain modification.

### CROSS-REFERENCE TO RELATED APPLICATIONS

This application claims priority to U.S. Provisional Application 63/548,971 filed on Feb. 2, 2024, which is incorporated herein by reference in its entirety.

### FIELD OF THE DISCLOSURE

The present disclosure is related to methods of inter-domain modification of a fungal population and modified bacteria for insertion of target sequences into fungal populations.

### SEQUENCE LISTING

The Instant Application contains a Sequence Listing which has been submitted electronically in XML format and is hereby incorporated by reference in its entirety. Said XML copy, created on Jan. 28, 2025, is named “SEQ_LIST—107668306-P230419US02.xml” and is 2,097 bytes in size. The Sequence Listing does not go beyond the disclosure in the application as filed.

### BACKGROUND

Conjugation is a naturally occurring form of horizontal gene transfer (HGT) that allows the exchange of genetic information among bacteria in the wild, which has also been used to engineer organisms in situ. Bacteria also conjugate with a variety of eukaryotic recipient cells, most commonly from bacterial donor Agrobacterium tumefaciens to plant cells. While A. tumefaciens is uniquely found to perform inter-domain conjugation (IDC) in the wild, other highly genetically tractable bacteria, such as Escherichia coli, can be modified to perform IDC with diatoms, mammalian cells, and multiple yeast species. And while the majority of microbiome research has focused on bacteria, fungi also play important roles, notably as pathogens such as Candida albicans, a leading cause of nosocomial infections.

Conjugative transfer of DNA occurs in multiple stages in the bacterial cell. First, a complex of proteins called the “relaxosome”, containing catalytic relaxases, nicks the conjugative plasmid at the origin of transfer (ori^{T}), and transfers one strand of the plasmid DNA to the membrane-bound type IV secretion system (T4SS). The T4SS transports the relaxosome-DNA complex through both bacterial membranes and a pilus connecting the donor and recipient cells. For E. coli T4SS, the DNA re-circularizes in the recipient cell to recreate the original plasmid.

Conjugation offers an opportunity to modify synthetic microbial consortia, and it has already been used for probiotics, defense against antibiotic-resistant pathogens, crop modification for desired traits, and control of undomesticated microbial species. IDC is currently limited as a tool for synthetic biology, however, by its relatively low efficiency. The vast majority of conjugation research has focused on lowering efficiency further, in an effort to prevent the spread of antibiotic resistance, which occurs through conjugative transfer of resistance-coding genes. Conjugation transfer terms between E. coli and the genetically tractable yeast species Saccharomyces cerevisiae are typically below 1 in 1,000 yeast cells, though recent work has succeeded in generating >10×DNA-transfer terms by selectively mutating the T4SS machinery. Another recent approach demonstrated increased conjugative efficiency between bacteria but used glass beads to colocalize donor and recipient cells, limiting its usefulness outside of laboratory settings. Since IDC recipients are also unable to propagate conjugative plasmids-unlike bacterial recipients which can act as conjugative donors, allowing logistic transconjugant growth-maximizing efficiency is crucial.

What is needed are systems and methods that allow bacteria to deliver target DNA to fungi, providing killing, gain of function, or otherwise altering recipient yeast populations.

### BRIEF SUMMARY

In an aspect, a method of inter-domain modification of a fungal population comprises co-culturing a bacterial population and the fungal population under growth conditions for the bacteria, wherein the bacterial population comprises a first bacterial plasmid comprising a bacterial selection marker and an operon encoding a type IV secretion system for conjugative transfer, and a second bacterial plasmid comprising a yeast selection marker, an expression cassette for expression of a transferred DNA sequence, an origin of transfer sequence for conjugative transfer of the second bacterial plasmid: or wherein the bacterial population comprises a single bacterial plasmid comprising a bacterial selection marker, a yeast selection marker, an expression cassette for expression of a transferred DNA sequence, an origin of transfer sequence for conjugative transfer of the single bacterial plasmid, and an operon encoding a type IV secretion system for conjugative transfer, and maintaining growth of the bacterial population during the co-culturing by controlling an essential nutrient for growth of the bacterial population, wherein the second bacterial plasmid or the single bacterial plasmid is transferred to at least a portion of the fungal population to provide the inter-domain modification.

In another aspect, a method of intra-domain killing of a fungal population in an infected host comprises administering a bacterial population to the host, wherein the bacterial population comprises a first bacterial plasmid comprising a bacterial selection marker and an operon encoding a type IV secretion system for conjugative transfer, and a second bacterial plasmid comprising a yeast selection marker, an expression cassette for a Cas9 nuclease, an expression cassette for expression of a guide RNA, and an origin of transfer sequence for conjugative transfer of the second bacterial plasmid, wherein no repair sequence for homology-directed repair of the Cas9-mediated double-strand break is provided: wherein the Cas9 nuclease and the guide RNA cut a target sequence in the fungal population, wherein the second bacterial plasmid is transferred to at least a portion of the fungal population to provide the intra-domain killing by cutting the target sequence in the fungal population.

In a further aspect, a modified bacteria comprises a first bacterial plasmid comprising a bacterial selection marker and an operon encoding a type IV secretion system for conjugative transfer, and a second bacterial plasmid comprising a yeast selection marker, an expression cassette for a Cas9 nuclease, an expression cassette for expression of a guide RNA, and an origin of transfer sequence for conjugative transfer of the second bacterial plasmid: wherein no repair sequence for homology-directed repair of the Cas9-mediated double-strand break is provided, and wherein the Cas9 nuclease and the guide RNA cut a target sequence in a fungal population.

In another aspect, a modified bacteria comprises a single bacterial plasmid comprising a bacterial selection marker, a yeast selection marker, an expression cassette for expression of a transferred DNA sequence, an origin of transfer sequence for conjugative transfer of the single bacterial plasmid, an operon encoding a type IV secretion system for conjugative transfer, and an expression cassette for a Cas9 nuclease, wherein no repair sequence for homology-directed repair of the Cas9-mediated double-strand break is provided, and wherein the Cas9 nuclease and the guide RNA cut a target sequence in a fungal population.

### BRIEF DESCRIPTION OF THE DRAWINGS

FIG. 1 is a schematic of conjugative transfer of DNA.

FIGS. 2A-D illustrate batch culturing cross-feeding populations revealing relationships between population ratios and trans-kingdom modification (IDC).

FIG. 2A shows experimental setup of batch cultures. Cells were combined in a 96-well microplate with varying levels of leucine (L) and tryptophan (W) in a minimal media cocktail. Co-cultures and monocultures are incubated at 30° C. with continuous shaking and measured for fluorescence of each species every 15 minutes. After 18-24 hours of growth, cells are diluted 1:10 into new media to continue growing. Simultaneously, a 1:10 dilution of cells is prepared for flow cytometry, and an undiluted 100 μL was plated onto modification-selective plates.

FIG. 2B shows compositional outcomes of co-cultures. Split heatmap of bacterial cell counts (light gray) and yeast cell counts (dark gray) for each co-culture pairing (columns) over a range of leucine and tryptophan (LW) concentrations (rows), from flow cytometry of batch culture day 6 (mean of four replicates). Counts were normalized to max cell counts per species—usually determined by input cell count, which is higher than carrying capacity—then multiplied uniformly to enhance color brightness, to better visualize low-density populations. “Cross” pertains to cross-feeding cells, “E” to Escherichia coli and “S” to Saccharomyces cerevisiae. At 0% LW, cross-feeding pairs' (E_{cross}-S_{cross}) growth is imperceptibly small, E_{cross}-S bacterial commensalism, E-S_{cross} competitive exclusion of yeast. Right: determination of donor-to-recipient ratio (“D:R”). Experimental results are for cis-donors. Brightness is mean of four replicates.

FIG. 2C shows normalized donor-to-recipient ratios correspond inversely to IDC counts. Ratios of normalized cell counts (cell count divided by maximum for that species across experiment) of bacterial donors and yeast recipients, calculated from flow cytometry data, plotted over time for each cell pairing, at 5% LW (left). Raw IDC counts from colony forming units (CFU) on selectable media for the same conditions and cell pairings (right). Lines represent means of four replicates.

FIGS. 2D and E show the correlation between donor-to-recipient ratios and IDC increases in time. 2D shows log-log distribution of donor-to-recipient ratio IDC counts for each cell pairing at day 7, for all LW %, with linear fits. 2E shows slopes of log-log plot linear fits for all days of batch culture, showing decreasing slope over time. Black bars=standard error of mean.

FIGS. 3A-C show mannose disruption of cell aggregates lowers inter-domain modification (IDC), interrupts bacterial commensalism.

FIG. 3A shows mannose interrupts mixed aggregates in culture. Microscopy images of batch co-culture after six days, either without (left) or with (right) mannose supplemented in media. Cells shown are trans-WT bacteria (E) and cross-feeding yeast (S_{cross}) at 15% LW, chosen to exemplify differences in clumping. Samples were diluted 1:10, imaged with a 10× objective. Scale bar=100 μm.

FIG. 3B shows interrupting clumps with mannose depresses IDC. Raw IDC counts (CFU) for samples at 15% LW without mannose supplementation (left column) are ≥ 10× those with mannose supplementation (right column). Lines represent means of three replicates.

FIG. 3C shows interrupting clumps with mannose prevents commensalism for cross-feeding E. coli. Normalized donor-to-recipient ratios for commensal E_{cross}-S pairing after six days of batch culturing, calculated from fluorescence data. Mannose-minus (hatched) and mannose-plus (open) samples show that clumping is required to sustain cross-feeding bacteria with WT yeast, especially at lower LW %. Bars represent mean of three replicates, error bars 95% CI, significance via two-sample/test, p=0.0010, 0.0041, 0.0071, and 0.018, for LW=0%, 5%, 10%, 15%, respectively.

FIGS. 4A-C show deterministic modeling shows limits of IDC transfer terms, proximity benefits, in and out of aggregates.

FIG. 4A shows IDC transfer-term sweep for “free” cell model. Heatmaps showing predicted number of transconjugants for a range of IDC transfer terms γ (y-axis) over six days of batch culturing, assuming cells are unable to clump, and thus conjugate via random collisions. Two of the four experimental conditions that yielded IDC counts >0 with mannose are shown (experimental CFU=cyan heat markers). The line at γ=0.004 represents literature prediction for enteric E. coli IDC transfer term, the white lines represent range of IDC-transfer term values matching experimental data, roughly between 7*10⁻⁶ and 4*10⁻⁵.

FIG. 4B shows IDC transfer-term sweep for “clumped” cell model. Heatmaps showing predicted number of transconjugants for a range of “free” IDC transfer terms γ (y-axis) and “clumped” IDC transfer terms γ_{c} (x-axis), for one representative experimental conditions at day six of batch culturing. White boxes represent experimental inter-domain modification IDC counts.

FIG. 4C shows proximity term sweep for “clumped” model. Heatmaps showing predicted bacterial fluorescence signal (color) for range of proximity-benefit multiplier P (y-axis) over six days, for E_{cross}-S condition at 0% LW. Gray horizontal bar shows approximate P value matching experimental co-culture data, i.e. a P value high enough (˜50) to allow E_{cross} growth solely from clumping to S.

FIGS. 5A-D show mixed colonies follow similar dynamics to culture conditions, show increased IDC for more spatially mixed populations.

FIG. 5A shows the experimental setup of colony assay. Cells are combined and pipetted in 2 μL droplets onto minimal media with 2% agar. Each plate contains ≥18 colony replicates of one cell pairing, and one amino acid concentration. After each days' growth, 6 colonies are imaged with a wide-field fluorescence microscope. 3 of these continue to be imaged daily, while the other 3 are scraped, washed, diluted for flow cytometry and IDC plating.

FIG. 5B shows an example of mixed colony cell distribution. Fluorescence microscopy images for each cell pairing at 100% LW (top row) and 0% LW (bottom row). All pairings here include cis-donors. Yeast are displayed in the bright channel, bacteria in dark. Channels are scaled for brightness to emphasize distribution, scale bar=1000 μm.

FIG. 5C shows colocalization showing divergent intermixing at 0% amino acids, with competitive exclusion driving spatial distribution. Li colocalization analyses of colonies (ICQ=0.5 is complete colocalization between channels, ICQ=−0.5 is complete spatial segregation) show range of outcomes for 0% LW colonies, less so for 100% LW colonies. All ICQ values range from −0.1-−0.5, implying competitive spatial segregation. Distributions shown are of cis-donor pairings. Calculated ICQ values for each replicate and condition represented by dots, 95% CI of the mean by vertical bars. Stars denote p-values from ANOVA 1-way test of 95% confidence between all 4 pairings using sum of squares test.

FIG. 5D shows colocalization correlates positively with IDC values. ICQ values plotted against raw IDC (CFU) counts for cis-donor pairings, at 0% and 100% LW, for IDC≥2. For the smaller range of ICQ values at 100% LW, IDC counts slow little divergence, whereas at 0% LW, IDC correlates positively with ICQ.

FIGS. 6A-D show utilizing population dynamics allows IDC-mediated rescue of unhealthy recipient populations.

FIG. 6A shows yeast growth is rescued by cross-feeding donor IDC. Yeast cell counts from flow cytometry, normalized to max count after day 1, for each cell pairing of S_{cross} at 10% UH, 0% L. In monoculture (dotted line), S_{cross} grows poorly at 10% UH. WT bacterial donors competitively exclude S_{cross} (dot-dash line), despite their ability to transfer IDC plasmid that would rescue recipients. E_{cross} donors, on the other hand, are able to transfer rescuing plasmid (solid line), allowing full S_{cross} rescue. Means of six replicates over two experiments shown as traces, shading as standard deviation.

FIG. 6B shows that batch culture growth in a rescue assay shows greater success for starved donors. Split heatmaps of normalized cell counts from flow cytometry for four cell pairings (columns) and four concentrations of uracil and histidine (rows). All samples grown with 0% leucine to starve E_{cross} (bacteria in dark gray). Yeast (light gray) auxotrophic for URA3 or HIS3, show greater growth upon receiving conjugated pTA-Mob 2.0 (cis), which is only significant when paired with E_{cross}. Brightness is mean of six replicates across two experiments, normalized to max cell count per species and experiment, multiplied uniformly to visualize low-density strains.

FIG. 6C shows model prediction of rescue phase map. Predictions for IDC counts based on clump model, adapted for rescue assay conditions. Concentrations of L (y-axis), U, and H (x-axis), for each cell pairing in rescue assay shown, with values 0%, 5%, 10% and 15% highlighted with white lines. Note that while rescue conditions don't include a range of leucine concentrations (only 0%), the model predicts a range of [L] over which E_{cross} could rescue yeast more effectively than WT bacteria.

FIG. 6D shows conceptual phase map of IDC outcomes. Comparing rates of bacterial (y-axis) and yeast (x-axis) growth, as controlled in rescue assay by amino acid levels. At low enough rates for both species, populations collapse before sufficient IDC can occur. When bacteria are sufficiently supplied with nutrients (or don't require them), competitive exclusion suppresses rescue of yeast by transconjugant growth. At low bacterial growth rates, but moderately low yeast growth rates, enough yeast cells are present to sustain growth of the starved bacteria for long enough to drive IDC, and the lack of competitive exclusion from the unhealthy donors allows for full rescue of recipient population.

FIGS. 7A-D show IDC-mediated CRISPR killing is able to drive recipient population extinction and is mannose-interruptible.

FIG. 7A shows design of IDC-mediated CRISPR system. pTA-Mob 1.0 T4SS plasmid (trans) is paired with a Cas9 plasmid that contains the ori^{T} sequence (allowing for transfer), HIS3 yeast selection marker, and sgRNA coding for a connector region in BFP-URA plasmid. Recipient yeast are Δura30. Δhis3::HPHMX6 and carry BFP-URA plasmid. Upon IDC transfer, BFP-URA plasmid is continually cut via Cas9, with no repair template for homology-directed repair, but yeast can continue to grow in media supplemented with uracil. In this way, IDC efficiency can be measured independently from CRISPR cutting efficiency, by plating for IDC (SC-H) and then replica plating for cut yeast (SC-UH). Finally, cut-verified donors are grown in batch culture with CRISPR recipient yeast at 0% U to gauge ability to depress recipient population through Inter-domain conjugation and genetic modification and subsequent killing.

FIG. 7B shows growth plots of co-cultures show IDC-mediated killing in some conditions. Fluorescence measurements, shown at the end of each day, from 12 days of batch culturing of cell pairs at four concentrations of tryptophan (rows). Trp-auxotrophic recipients (S_{cross}) collapse for both pairings (cutting donor and no-ori^{T} negative control donor) at 1% W, but only for the cutting donor at 5% W. Lines are means of three replicates, shaded region standard deviation. Mannose was added to experiment after day 6, shown as vertical dotted lines.

FIG. 7C shows comparing recipient population decline between co-cultures and monoculture. Fold-decreases in yeast growth, based on normalized fluorescence, from yeast in co-culture to yeast monoculture. Fold decrease=−(Monoculture Citrine)/(Co-culture Citrine). Points are means of three replicates, bars 95% CI. Stars represent p-value significance from two-sample t-test, with no significance for time points lacking stars (p>0.05). Vertical dotted lines designate addition of mannose at day 6.

FIG. 7D shows transient IDC counts show rise and subsequent, mannose-initiated fall. IDC counts (CFU) for both cell pairings (negative control donor, triangles, is unable to transfer DNA, all counts=0), over four W % (rows). After addition of mannose (vertical dotted line), most surviving co-cultures drop in IDC counts. Note that 5% W yeast population is coincidentally driven to extinction near day 6, and thus unaffected by mannose. IDC counts here are “transient” because transconjugants are terminal at 0% U, so transconjugants are unable to persist in co-culture across days.

FIG. 8 shows scatterplots relating bacterial growth rate (Rb), yeast growth rate (Ry), and the ratio of yeast to bacterial growth rate (Ry/Rb) to inter-domain modification.

The above-described and other features will be appreciated and understood by those skilled in the art from the following detailed description, drawings, and appended claims.

### DETAILED DESCRIPTION

Described herein are systems and methods that allow bacteria to deliver target DNA to fungi, providing killing or otherwise altering recipient yeast populations. In specific aspects, described herein are strains of E. coli and S. cerevisiae mutated to allow tunable population control via engineered cross-feeding between E. coli and S. cerevisiae, in which each species is auxotrophic for an essential amino acid that the other species overproduces. This approach also has implications in colony settings, where conjugation events between two spatially constrained populations occurs along population boundaries, and mutualism between cells can greatly increase intermixing of populations in both bacteria and yeast, hypothetically creating more population boundaries along which IDC can occur. Unexpectedly, when bacterial populations were kept low; transfer rates increased. Without being held to theory, it is believed that mitigating competitive effects on the recipient yeast population, which has a lower growth rate, facilitates transfer.

Also described herein is a novel conjugation-mediated CRISPR killing system. Plasmid DNA transferred to recipients in this system includes Cas9 and a guide sgRNA targeting a yeast plasmid carrying an essential gene. This yeast plasmid may be assembled via a widely-used Yeast Toolkit (YTK) or other common cloning techniques, and thus could contain a wide range of functional genes, and the target cut site is in a generic sequence that would be compatible with alternative assemblies. Moreover, modifying the sgRNA to target other (gDNA or episomal) sequences allows a wide range of possible functions. Specifically, the YTK plasmid contains blue fluorescent protein (BFP) and URA3, an essential gene for the biosynthesis of uracil in yeast. Once cut, yeast recipients are rendered auxotrophic for uracil, and are terminal in media lacking uracil. Conveniently, targeting a nongenomic DNA sequence allows measurement of efficiencies of DNA-transfer and CRISPR cutting separately, by growing yeast cells in media with- or without uracil. Moreover, because the target plasmid contains BFP, cutting can be measured fluorescently as well.

Further, it was determined that conjugative transfer primarily occurs after bacteria bind to mannoproteins ubiquitous in the yeast cell wall, and that therefore this action can be interrupted by adding mannose to media, which saturates mannose-binding receptors in the bacterial donors. In experiments described herein, a (slow-growing) recipient yeast population was conjugatively collapsed, and ongoing killing action was interrupted by adding mannose several days into batch co-cultures of donors and recipients.

Advantages of the compositions and methods described herein are as follows. First, there appears to be no existing inter-domain modification system capable of killing or otherwise altering recipient fungal populations. Research on this system typically focuses on obtaining selectable (single) transconjugants, for purposes similar to cloning, and not population-wide outcomes. Whereas in bacteria, for which conjugation, and phage treatment can alter populations, similar tools for fungal members of microbiomes are lacking. And while much research of late has focused on protein transfer to eukaryotic cells via a similar Type VI Secretion System, utilizing DNA—which can be easily engineered and tailored to a desired recipient species—is a much more versatile option for affecting recipient functions.

Second, while conjugation between these species has been established, tuning populations to maximize conjugative transfer has not been demonstrated in this way. Importantly, this strategy relies only on engineering of the donor strain.

Third, a CRISPR system for yeast that utilizes a Cas9 and an oriT sequence that allows for conjugative transfer of the entire CRISPR system.

Fourth, the sgRNA sequence targeting a modular yeast toolkit plasmid allows novel versatility for laboratory and synthetic biology research that uses Saccharomyces cerevisiae, since the toolkit allows for easy changes in functional genes carried on the recipient plasmid, and the cut target is agnostic to genes introduced in this way.

And fifth, the demonstration of mannose-reversibility in conjugative transfer to fungal recipients allows more control over synthetic consortia between these two species, and there is an opportunity to adapt this system to other fungal recipient species, since mannoproteins exist in the cell walls of many such species.

Several yeast species can be pathological, and there are few treatments for such diseases. For example, biofilm-forming yeast in the genus Candida are responsible for greater than 10% of nosocomial infections, and can be lethal. Moreover, many fungal commensals can overgrow and exacerbate other illnesses such as ulcers and gut dysbiosis, often worsened by administration of (bacterial) antibiotics. Unlike bacterial infections, for which we have a wide range of antibiotics, phage treatments, and a growing body of knowledge of microbiome bacterial composition, we have very few antifungal treatments, no corresponding phage-like treatment, and woefully limited insight into fungal roles in microbiomes. The compositions and methods described herein can be quickly engineered to modify or kill specific fungal recipients, offering an important step toward targeted microbiome engineering.

In an aspect, a method of inter-domain modification of a fungal population, comprises co-culturing a bacterial population and the fungal population under growth conditions for the bacteria, wherein the bacterial population comprises a first bacterial plasmid comprising a bacterial selection marker and an operon encoding a type IV secretion system for conjugative transfer, and a second bacterial plasmid comprising a yeast selection marker, an expression cassette for expression of a transferred DNA sequence, and an origin of transfer sequence for conjugative transfer of the second bacterial plasmid; and maintaining growth of the bacterial population during the co-culturing by controlling an essential nutrient for growth of the bacterial population, wherein the yeast growth rate divided by bacterial growth rate is greater than 0.3, and wherein the second bacterial plasmid is transferred to at least a portion of the fungal population to provide the inter-domain modification. In an aspect, the yeast growth rate divided by bacterial growth rate is greater than 0.3.

Genes that may be included in an operon encoding a type IV secretion system for conjugative transfer include:

| Gene | Function |
|---|---|
| KlaA | Host lethality, fertility inhibition (kilA locus) |
| KlaB | Host lethality, fertility inhibition (kilA locus) |
| KlaC | Host lethality, fertility inhibition (kilA locus) |
| KleA | Host toxicity |
| KleB | Host toxicity |
| KleC | Host toxicity |
| KleD | Plasmid maintenance |
| KleE | Plasmid maintenance |
| KleF | Plasmid maintenance |
| KorC | Global transcription repressor |
| KlcB | Inhibition of KorA, derepression of korA-targeted gene(s) |
| trfA | Replication initiation |
| TrbA | Global transcription repressor |
| ssb | Single stranded binding protein |
| KlbA | Host toxicity |
| TrbB | T4SS protein, membrane anchor |
| TrbC | P-type propilin |
| TrbD | Pilus assembly |
| TrbE | T4SS ATPase |
| TrbF | Pilus assembly |
| TrbG | T4SS protein, membrane anchor |
| TrbH | T4SS protein, membrane anchor |
| TrbI | T4SS protein, membrane anchor |
| TrbJ | T4SS protein, membrane anchor |
| TrbK | Entry exclusion protein |
| TrbL | Mating pair formation |
| TrbM | Mating pair formation |
| TrbN | Mating pair formation |
| TrbO | Transmembrane protein |
| TrbP | Putative pilus acetylase |
| upf31.7 | Unknown function |

An origin of transfer sequence for conjugative transfer or ori^{T} is a noncoding region of bacterial DNA that is both a substrate and recognition sequence for relaxase proteins which nick the conjugative plasmid at the ori^{T}, and transfer one strand of the plasmid DNA to the membrane-bound type IV secretion system (T4SS). An exemplary ori^{T} is the ˜100 bp region from the incompatibility type-P (“IncP”) conjugative plasmid first isolated from Pseudomonas aeruginosa (aka RP4, RK2), with sequence gggcaggataggtgaagtaggcccacccgcgagcgggtgttccttcttcactgtcccttattcgcacctggcggtgctcaacgggaat cctgctctgcgaggctggccgg (SEQ ID NO: 1).

Conjugative transfer of DNA occurs in multiple stages in the bacterial cell. Thus, the system includes both an origin of transfer sequence for conjugative transfer and an operon encoding a type IV secretion system for conjugative transfer. First, a complex of proteins called the “relaxosome”, containing catalytic relaxases, nicks the conjugative plasmid at the origin of transfer (ori^{T}), and transfers one strand of the plasmid DNA to the membrane-bound type IV secretion system (T4SS). The genes for the relaxase operon include, for example, the Tra1 region, including relaxase operon TraI-H, which together nick and bind to the ori^{T}. The T4SS transports the relaxosome-DNA complex through both membranes and a pilus connecting the donor and recipient cells. For E. coli T4SS, the DNA re-circularizes in the recipient cell to recreate the original plasmid.

The regions Tra1 and Tra2 transfer plasmid DNA. Tra1 includes genes encoding the relaxase (traH-J), primase (traA-G), and leader (traK-M) operons which mobilize the plasmid to the recipient. The relaxase and leader operon encode the relaxosome. Assembly of the protein complex (TraH-J) is initiated by TraJ binding to the 19-bp inverted repeat sequence in the oriT. After formation of the relaxosome, TraI nicks and covalently binds to the plasmid DNA, ready for transfer to the recipient cell. The primase operon also includes the TraG protein, which couples DNA processing by the relaxosome to DNA transfer by delivering the protein-DNA complex to the mating pair formation proteins. The Tra2 region contains proteins (TrbB-L and TraF) required for mating pair formation, many of which are associated with the cell membrane. TrbC encodes a peptide responsible for forming the pilus. The pilus allows initial contact between the two cells and enables the transfer of single-stranded plasmid DNA to the recipient cell.

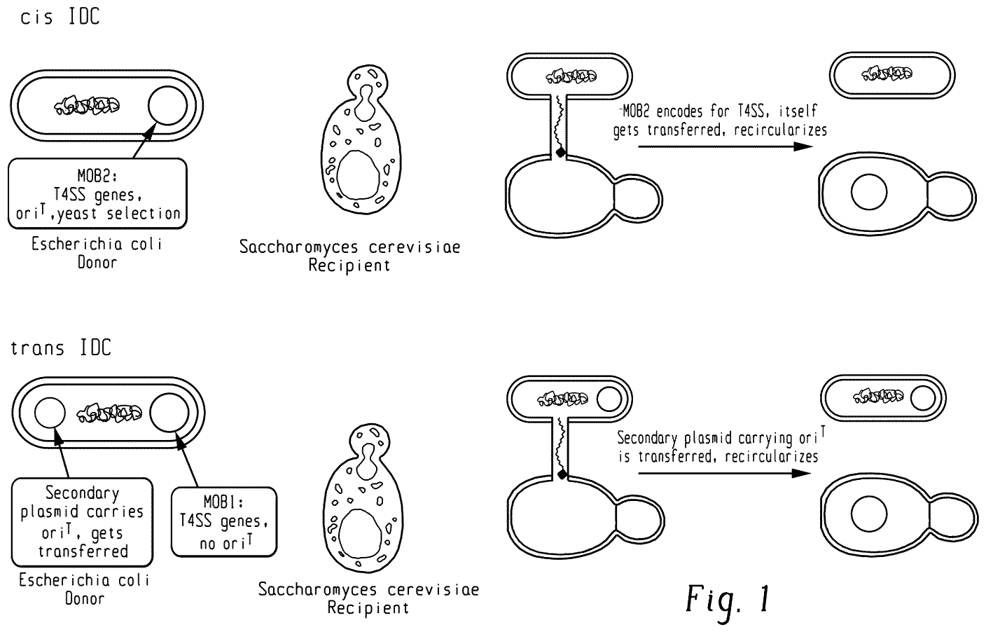

In the aspect wherein the bacteria include a first and second bacterial plasmid, the conjugation is trans conjugation, in which the ori^{T} is on a separate plasmid, which is transferred (FIG. 1).

In another aspect, a method of inter-domain modification of a fungal population, comprises co-culturing a bacterial population and the fungal population under growth conditions for the bacteria, wherein the bacterial population comprises a single bacterial plasmid comprising a bacterial selection marker, a yeast selection marker, an expression cassette for expression of a transferred DNA sequence, an origin of transfer sequence for conjugative transfer of the single bacterial plasmid, and an operon encoding a type IV secretion system for conjugative transfer; and maintaining growth of the bacterial population during the co-culturing by controlling an essential nutrient for growth of the bacterial population, wherein the yeast growth rate divided by bacterial growth rate is greater than 0.3, wherein the single bacterial plasmid is transferred to at least a portion of the fungal population to provide the inter-domain modification.

In the aspect wherein the bacteria include a single bacterial plasmid, the single plasmid carrying the relaxosome genes itself contains an ori^{T} and thus is transferred to a recipient cell (FIG. 1). This is the cis method of conjugation.

As illustrated in FIG. 1, the inter-domain modification method described herein can proceed via a cis (single bacterial plasmid) or a trans (two bacterial plasmids) conjugation.

An exemplary bacterium is E. coli, though P. aeruginosa, Bacillus subtilis, Bartonella henselae, and Rhizobiales sp. could serve as donors. Exemplary yeast species for the methods described herein include Saccharomyces sp., Lachancea sp., Kluyveromyces sp., Pichia sp., Candida sp., Malassezia sp., Aspergillus sp., or Yarrowia sp. Exemplary yeast include S. cerevisiae, Candida glabrata, Malassezia restricta, or Aspergillus fumigatus.

Advantageously, the co-culturing methods described herein can be done in batch or continuous culture. Also as used herein, co-culturing includes culturing on solid surfaces in addition to culturing in solution.

Advantageously, in the method described herein, the growth of the bacterial population during the co-culturing is maintained by controlling an essential nutrient for growth of the bacterial population. In an aspect, maintaining growth of the bacterial population during the co-culturing by controlling an essential nutrient for growth of the bacterial population can comprise limiting the concentration of the essential nutrient in the co-culture and providing the essential nutrient by overproduction of the essential nutrient from the fungal population, herein the bacterial population is auxotrophic for the essential nutrient. In a further aspect, the bacterial population overproduces a second essential nutrient, and the fungal population is auxotrophic for the second essential nutrient, and wherein the method comprises limiting the second essential nutrient in the co-culture.

Any other means of limiting bacterial growth relative to yeast growth, whether through bacterial toxins, limiting essential nutrients, altering the metabolic interactions between species in co-culture, or in any other way establishing growth conditions that disfavor the bacterial growth rate relative to other species in co-culture.

In an aspect, the essential nutrient is an amino acid such as leucine, tryptophan or histidine, or a nucleoside such as uracil.

In an important aspect, the inventors found that controlling the population of the bacteria in the culture is important to mitigate negative effects on the growth of fungus and increase transfer rates. Extensive modeling has been performed, and it was found that when the yeast growth rate divided by bacterial growth rate is greater than 0.3, transfer rates can be maximized.

In an aspect, the yeast growth rate (dY/dt) and the bacterial growth rate (dB/dt) are calculated by:

$$Bacterial growth : \frac{dB}{dt} = {R}_{b} * B ( 1 - \frac{B}{{K}_{b}} - \frac{{c}_{y} Y}{\text{?}} )$$

$$Yeast ( non conjugated ) growth : \frac{dY}{\text{?} t} = \text{?} * Y ( \begin{matrix}1 & \begin{matrix}Y \\ \text{?}\end{matrix} & \begin{matrix}{c}_{b} B \\ \text{?}\end{matrix} ) \text{?}\end{matrix} ( \begin{matrix}B * Y \\ B + Y\end{matrix} )$$

$$\text{?} \text{indicates text missing or illegible when filed}$$

Wherein
R_{b}=free growth rate of bacteria,
K_{b}=Bacterial carrying capacity,
K_{y}=Yeast carrying capacity,
B=starting number of bacteria cells,
Y=starting number of yeast cells,
c_{y}=Ecological niche overlap (effect of yeast on bacteria),
R_{y}=free growth rate of yeast, and
c_{b}=Ecological niche overlap (effect of bacteria on yeast).

In alternative aspect, the yeast growth rate (dY/dt) and the bacterial growth rate (dB/dt) can be determined using means of controlling the relative growth rates of bacteria and yeast in coculture, whether by dosing antibiotics or antifungals (respectively), limiting essential nutrients for each, or changing pH or any other aspect of coculture media to favor one species over the other, and to thereby determine the optimal growth rates for IDC transfer.

In yet another aspect, it was found that conjugative transfer primarily occurs after bacteria bind to mannoproteins ubiquitous in the yeast cell wall, and that therefore this action can be interrupted by adding mannose to media, which saturates mannose-binding receptors in the bacterial donors. In an aspect, the method comprises adding 4% w/v mannose to the co-culture to reduce a percentage of fungi undergoing inter-domain modification.

Virtually any DNA sequence can be transferred using the methods described herein. Exemplary transferred DNA sequences include a gene encoding a metabolic enzyme, a gene encoding a drug resistance marker, a gene encoding a fluorescence marker, a virulence-modifying gene, or any genome-integrating or genome-editing machinery.

In an aspect, when the bacterial population comprises the first and second bacterial plasmid, the second bacterial plasmid can comprise an expression cassette for a Cas) nuclease, wherein the transferred DNA sequence expresses a guide RNA, wherein the fungal population comprises a target sequence for the guide RNA, wherein no repair sequence for homology-directed repair of the Cas9-mediated double-strand break is provided wherein the Cas9 nuclease and the guide RNA cut the target sequence in the fungal population. In a specific aspect, the target sequence in the fungal population is in an essential yeast gene, and wherein cutting the target sequence kills the yeast.

In another aspect, when the bacterial population comprises a single bacterial plasmid, the single bacterial plasmid can comprise an expression cassette for a Cas9 nuclease, wherein the transferred DNA sequence expresses a guide RNA, wherein the fungal population comprises a target sequence for the guide RNA, wherein no repair sequence for homology-directed repair of the Cas9-mediated double-strand break is provided, wherein the Cas9) nuclease and the guide RNA cut the target sequence in the fungal population

In a further aspect, a method of intra-domain killing of a fungal population in an infected host comprises administering a bacterial population to the host, wherein the bacterial population comprises a first bacterial plasmid comprising a bacterial selection marker, and a second bacterial plasmid comprising a yeast selection marker, an expression cassette for a Cas9 nuclease, an expression cassette for expression of a guide RNA, and an origin of transfer sequence for conjugative transfer of the second bacterial plasmid: wherein no repair sequence for homology-directed repair of the Cas9-mediated double-strand break is provided, and wherein the Cas9 nuclease and the guide RNA cut a target sequence in the fungal population, wherein the second bacterial plasmid is transferred to at least a portion of the fungal population to provide the intra-domain killing by cutting the target sequence in the fungal population. In an aspect, cutting the target sequence in the fungal population inactivates an essential gene in the fungal population. In an aspect, the essential gene is in a yeast plasmid. In an aspect, the target sequence is a non-genomic DNA sequence.

In an aspect, the host is infected with a biofilm-forming Candida sp. Such fungi are responsible for greater than 10% of nosocomial infections and can be lethal. Fungal commensals can also overgrow and exacerbate other illnesses such as ulcers and gut dysbiosis, and such infections can be exacerbated by administration of antibiotics.

The bacterial population can be administered to the host by any means suitable to administer bacteria for controlling infection. Administration can be oral such as in the form of a probiotic composition, parenteral, or by topical administration, for example.

Also described herein is a modified bacteria comprising a first bacterial plasmid comprising a bacterial selection marker, and a second bacterial plasmid comprising a yeast selection marker, an expression cassette for a Cas9 nuclease, an expression cassette for expression of a guide RNA, and an origin of transfer sequence for conjugative transfer of the second bacterial plasmid: wherein the Cas9 nuclease and the guide RNA cut the target sequence in the fungal population, but no repair sequence for homology-directed repair is provided. In an aspect, cutting the target sequence in the fungal population inactivates an essential gene in the fungal population. In an aspect, the essential gene is in a yeast plasmid. In an aspect, the target sequence is a non-genomic DNA sequence. In an aspect, the target sequence is a genomic DNA sequence.

In another aspect, a modified bacteria comprises a single bacterial plasmid comprising a bacterial selection marker, a yeast selection marker, an expression cassette for expression of a transferred DNA sequence, an origin of transfer sequence for conjugative transfer of the single bacterial plasmid an operon encoding a type IV secretion system for conjugative transfer, and an expression cassette for a Cas9 nuclease, wherein no repair sequence for homology-directed repair of the Cas9-mediated double-strand break is provided, and wherein the Cas9 nuclease and the guide RNA cut a target sequence in a fungal population.

CRISPR/Cas9 is a ribonucleoprotein (RNP) complex. CRISPR RNA (crRNA) includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

Guide RNA, or gRNA, can be in the form of a crRNA/tracrRNA two guide system, or an sgRNA single guide RNA. The guide RNA is capable of directing Cas9-mediated cleavage of target DNA. A guide RNA thus contains the sequences necessary for Cas9 binding and nuclease activity and a target sequence complementary to a target DNA of interest (protospacer sequence).

As used herein, a guide RNA protospacer sequence refers to the nucleotide sequence of a guide RNA that binds to a target genomic DNA sequence and directs Cas9 nuclease activity to a target DNA locus. In some embodiments, the guide RNA protospacer sequence is complementary to the target DNA sequence. “Complementary” or “complementarity” refers to specific base pairing between nucleotides or nucleic acids. Base pairing between a guide RNA and a target region can be via a DNA targeting sequence that is perfectly complementary or substantially complementary to the guide RNA. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the targeted sequence includes a protospacer adjacent motif (PAM) adjacent to the protospacer region which is a sequence recognized by the CRISPR RNP as a cutting site. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs.

A “Cas9” polypeptide is a polypeptide that functions as a nuclease when complexed to a guide RNA, e.g., an sgRNA or modified sgRNA. That is, Cas9 is an RNA-mediated nuclease.

As described herein, the Cas9 is a S. pyogenes Cas9 variant

The bacterial population can be administered in the form of a pharmaceutical composition. Pharmaceutical compositions include reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

Tablets and capsules for oral administration may be in unit dose form, and may contain conventional excipients such as binding agents, for example syrup, acacia, gelatin, sorbitol, tragacanth, or polyvinyl-pyrrolidone: fillers for example lactose, sugar, maize-starch, calcium phosphate, sorbitol or glycine: tabletting lubricant, for example magnesium stearate, talc, polyethylene glycol or silica: disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats: emulsifying agents, for example lecithin, sorbitan monooleate, or acacia: non-aqueous vehicles (which may include edible oils), for example almond oil, fractionated coconut oil, oily esters such as glycerine, propylene glycol, or ethyl alcohol: preservatives, for example methyl or propyl p-hydroxy benzoate or sorbic acid, and if desired conventional flavoring or coloring agents.

For topical application to the skin, the drug may be made up into a cream, lotion or ointment. Cream or ointment formulations which may be used for the drug are conventional formulations well known in the art. Topical administration includes transdermal formulations such as patches.

For topical application to the eye, the inhibitor may be made up into a solution or suspension in a suitable sterile aqueous or non aqueous vehicle. Additives, for instance buffers such as sodium metabisulphite or disodium edeate: preservatives including bactericidal and fungicidal agents such as phenyl mercuric acetate or nitrate, benzalkonium chloride or chlorhexidine, and thickening agents such as hypromellose may also be included.

In one embodiment, the pharmaceutically acceptable excipient is suitable for parenteral administration. Alternatively, the pharmaceutically acceptable excipient can be suitable for subcutaneous, intravenous, intraperitoneal, intramuscular, or sublingual administration. Pharmaceutically acceptable excipients include sterile aqueous solutions or dispersions and sterile powders for the extemporaneous preparation of sterile injectable solutions or dispersions. The use of such media and agents for pharmaceutically active substances is well known in the art.

Parenteral pharmaceutical compositions are typically sterile and stable under the conditions of manufacture and storage. The pharmaceutical composition may be in lyophilized form. The composition can be formulated as a solution, microemulsion, liposome, or other ordered structure suitable to high drug concentration. The excipient can be a solvent or dispersion medium containing, for example, water, ethanol, polyol (for example, glycerol, propylene glycol, and liquid polyethylene glycol), and mixtures thereof. A stabilizer can be included in the pharmaceutical composition.

Pharmaceutical compositions can include isotonic agents, for example, sugars, polyalcohols such as mannitol, sorbitol, or sodium chloride. Prolonged absorption of the injectable compositions can be brought about by including in the composition an agent which delays absorption, for example, monostearate salts and gelatin. The inhibitor can be formulated in a time release formulation, for example in a composition which includes a slow release polymer. The inhibitor can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

The inhibitor may be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the inhibitor can either be suspended or dissolved in the vehicle. Advantageously, adjuvants such as a local anaesthetic, preservative, and buffering agents can be dissolved in the vehicle. Subcutaneous administration can be daily administration.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Methods

Strain and plasmid construction: Yeast cells were derived from W303 strains developed in the art. Genetic drift opposes mutualism during spatial population expansion. Cross-feeding yeast strains (“S_{cross}”, yMM1430) have additional mutations to make them auxotrophic for tryptophan and leucine-overproducing: LEU4^{FBR} trp2Δ::NATMX4 prACTIyCerulean-tADHI@URA3, with leucine feedback resistance (FBR) resultant from deletion of codon 548 of LEU4. S_{cross} is also constitutively fluorescent for ymCitrine and yCerulean, whereas the baseline yeast used here (aka “S”, yMM1636) is only ymCitrine-fluorescent. Further mutations were introduced into these strains to make them auxotrophic for uracil and/or histidine, for IDC selection and CRISPR assay. Uracil was knocked out by amplifying a cassette of URA3 homology arms, transforming into yMM1430, and selecting for growth on 5-Fluoroorotic acid (5FOA). HIS3 was replaced with either KANMX6 or HPHMX6, depending on the strain (see Table 1 for list of strains and related experiments), by amplifying either resistance gene with overlap for HISMX6 regions.

TABLE 1
Strains used in this study

| | | Genetic | | | |
|---|---|---|---|---|---|
| ID | Species | features | Fluorescence | Source | Figures |
| yMM1585 | Yeast | Leu⁺⁺, Trp⁻, | ymCitrine, | 1, this | 2, 5, 6, |
| | | Ura⁻ | yCerulean | work | |
| yMM1636 | Yeast | His⁻ | ymCitrine | 1, this | All but 7 |
| | | | | work | |
| yMM1720 | Yeast | Leu⁺⁺, Trp⁻, | ymCitrine, | 1, this | 3, 4 |
| | | His⁻ | yCerulean | work | |
| yMM1786 | Yeast | Leu⁺⁺, Trp⁻, | ymCitrine, | 1, this | 7 |
| | | Ura⁻, His⁻ | yCerulean | work | |
| kMM0011 | Bacterium | None | None | 7 | All but 7 |
| kMM0127 | Bacterium | Trp⁺⁺, Leu⁻ | None | 7, this | All |
| | | | | work | |

Bacterial strains were derived from Keio Collection strains of single-gene knockouts, based on BW25113 background. Construction of Escherichia coli K-12 in-frame, single-gene knockout mutants: F-Δ(araD-araB)567 lacZ4787Δ::rmB-3λ-rph-1 Δ(rhaD-rhaB)568 hsdR514). WT E. coli (“E”) strains were BW25113, or Coli Genetic Stock Center (CGSC) #7636, containing different plasmids depending on the experiment (see Table 2 for list of plasmids and corresponding experiments). Cross-feeding mutations were introduced into CGSC #11110 (ΔtrpR789:kan^{R}), which lacks the trp repressor gene, and has been shown to be tryptophan-overproducing. Briefly, the kanamycin resistance gene at the trpR locus was “flipped” out via flippase recognition target (FRT) sequences and flippase-expressing plasmid pMM0821. Leucine auxotrophy was introduced by λred recombination of PCR-amplified ΔleuA781::kan^{R}, from CGSC #8373, using pMM0820, which expresses genes for λred. kan^{R} was again flipped out to obtain kMM127, a double knockout of ΔtrpR, ΔleuA, with no antibiotic resistance. Note that E_{cross} was originally constructed from ΔleuA::kan^{R} (CGSC #8373), but it caused severe aggregation in co-culture, such that cells would precipitate out of media immediately, whereas the same mutation introduced from the ΔtrpR::kan^{R} strain did not produce this result. Moreover, ΔleuB::kan^{R} (CGSC #11943) proved prototrophic for leucine over long time periods, despite its similar function in the leucine biosynthesis pathway.

TABLE 2
Plasmids used in this study

| ID | Species | Function/Features | Fluorescence | Source | Figures |
|---|---|---|---|---|---|
| pMM0819 | Bacterium | pProD-mCherry | mCherry | Addgene 87144 | All |
| pMM0820 | Bacterium | λ_{red} genes | None | 8 | None |
| pMM0821 | Bacterium | Flippase | None | 8 | None |
| pMM0892 | Bacterium | T4SS genes, Gent^{R} | None | pTA-Mob 1.0⁹ | 3, 4, 7 |
| pMM0893 | Both | T4SS genes, Gent^{R}, | None | pTA-Mob 2.0, | 1, 2, 5, 6 |
| | | URA3, HIS3, | | Addgene 149662 | |
| | | CEN/ARS, ori^{T} | | | |
| pMM1340 | Bacterium | sgRNA assembly | GFP | Addgene 90516 | None |
| | | vector, sfGFP | | | |
| pMM1341 | Bacterium | Cas9, sfGFP | GFP | Addgene 90519 | None |
| pMM1342 | Bacterium | sgRNA assembly, | None | This work | None |
| | | YTK target | | | |
| pMM1360 | Yeast | yeBFP, URA3, | BFP | 10, this work | 7 |
| | | CEN/ARS | | | |
| pMM1437 | Both | yeBFP, ori^{T}, HIS3, | BFP | 10, this work | 3, 4 |
| | | CEN/ARS | | | |
| pMM1438 | Both | Cas9, ori^{T}, sgRNA | None | This work | 7 |
| | | (YTK), HIS3 | | | |
| pMM1439 | Both | Cas9, sgRNA (YTK), | None | This work | 7 |
| | | HIS3 | | | |

IncP-type IDC plasmids pTA-Mob 1.0 (trans-transferring) and pTA-Mob 2.0 (cis-transferring) were used. pTA-Mob 2.0 contains gentamicin resistance for bacterial selection, URA3 and HIS3 genes for yeast selection, CEN6/ARSH4 for yeast maintenance, and the ori^{T} sequence required for conjugative transfer of the plasmid into recipients, whereas pTA-Mob 1.0 only carries gentamicin resistance. Constitutive bacterial reporter pMM0819 contains pProD:mCherry, using a synthetic reporter meant to be high-expressing and minimally susceptible to cell phase. IDC plasmids for trans-transfer were constructed using the Golden Gate-based Yeast MoClo Toolkit (YTK), to modularly assemble a fluorescent yeast reporter (pTDH3-yeBFP), IDC selection (HIS3), and yeast replication machinery (CEN6/ARSH4). The ori^{T} sequence was then added to the connector sequence downstream of yeBFP via Gibson assembly.

For IDC-mediated CRISPR killing assay, the S_{cross}, ura3Δ0 hismx6Δ::HPHMX6 strain yMM1786 was transformed with a plasmid containing pTIH3-yeBFP URA3 CEN/ARS. sgRNAs were designed to cut within the connector region of this plasmid (ConR1 from YTK), downstream of yeBFP, such that any YTK-assembled plasmid containing the ConR1 sequence could be a target in future experiments. CRISPR plasmids were assembled using. Briefly, oligos for five sgRNA sequences targeting the ConR1 region were designed using Benchling, PNK-phosphorylated and annealed. Annealed oligos were then assembled into sgRNA entry vector pMM1340 via Golden Gate assembly and transformed into bacteria, selecting with carbenicillin. Purified and sequence-verified sgRNA plasmids were then digested with EcoRV to isolate the sgRNA sequences with homology arms matching the insertion site of the Cas9 plasmid. Plasmid pMM1341, which contains Cas9, GFP, and HIS3, was digested with BsmBI to remove GFP and leave homology arms for sgRNA at each end of the resultant linear DNA. The two pieces were combined via yeast recombinant cloning. Finally, ori^{T} was inserted by ligating a modified version of the ori^{T} sequence with the assembled Cas9-sgRNA after digesting with AatII and SacII.

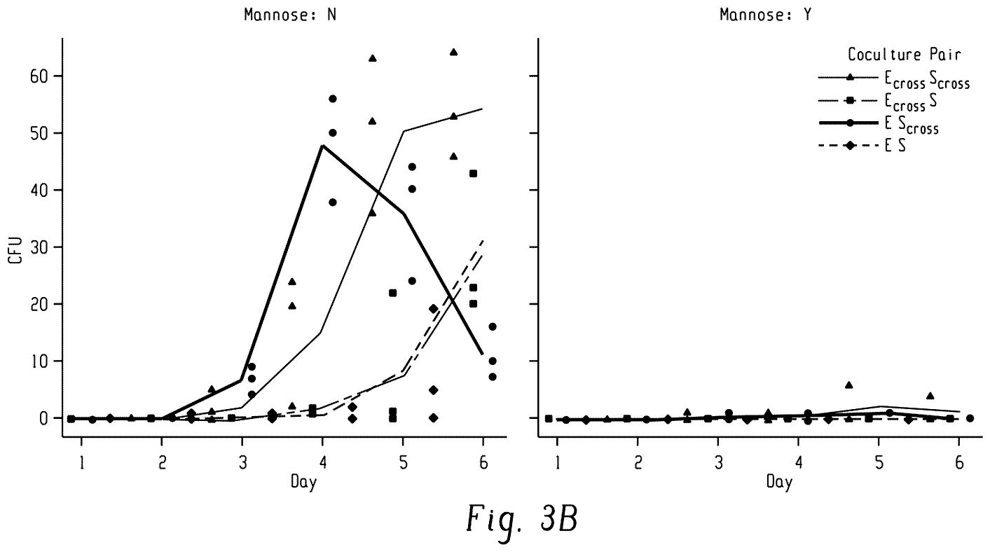

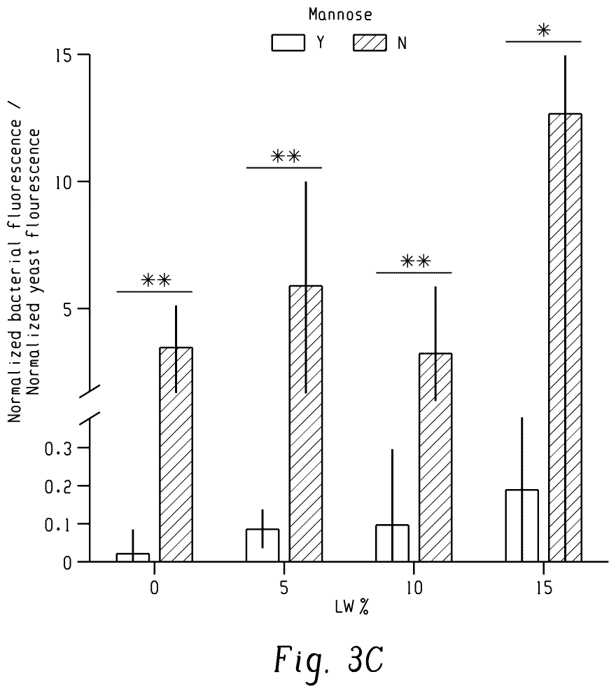

Batch culture experiments and IDC counting: Yeast and bacterial cultures used in each batch culture experiment were grown overnight in selective YPD or LB media, at 30° C. or 37° C., respectively. After ≥16 hours growth, bacterial strains were measured for OD600, yeast strains were measured for OD660, and each culture was washed at least 2 times with SC or M9 sans glucose or amino acids. Cells were then combined such that each reaction started with 1E7 cells, based on OD measurements. Growth media was composed of 200 μL of a 75:25 mixture of SC: M9 minimal media with 2% glucose, appropriate amino acids, and antibiotics to maintain each bacterial plasmid. Amino acid percentages in the text are based on the following molarities, considered 100%: L=762 μM, W=245 μM, U=178 μM, H=95.4 μM. For clumping experiment (FIG. 3), half of the media was supplemented with 4% mannose. Upon spiking cells into 96-well CellVis back-walled optical glass-bottom plates (cat #P96-1-N), plates were sealed with gas permeable membranes (Fisher Scientific cat #50-550-304) seals to allow air flow for aerobic conditions.

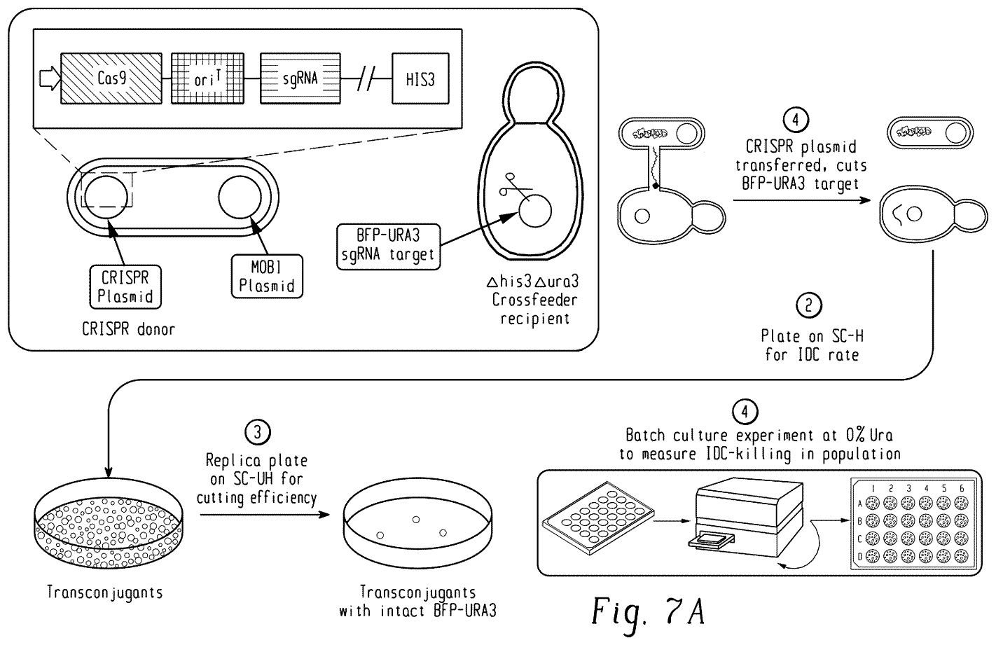

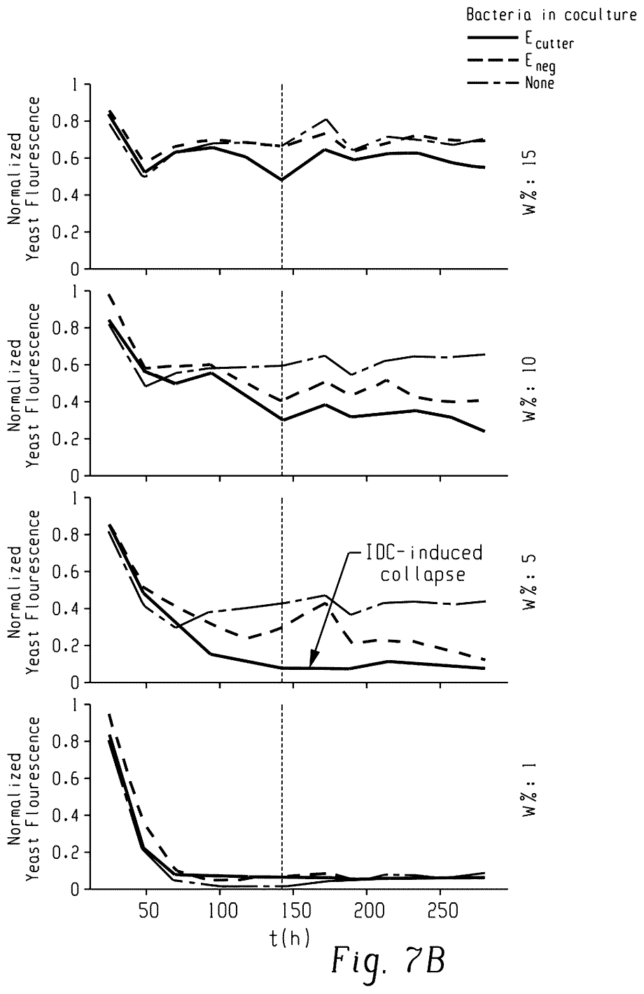

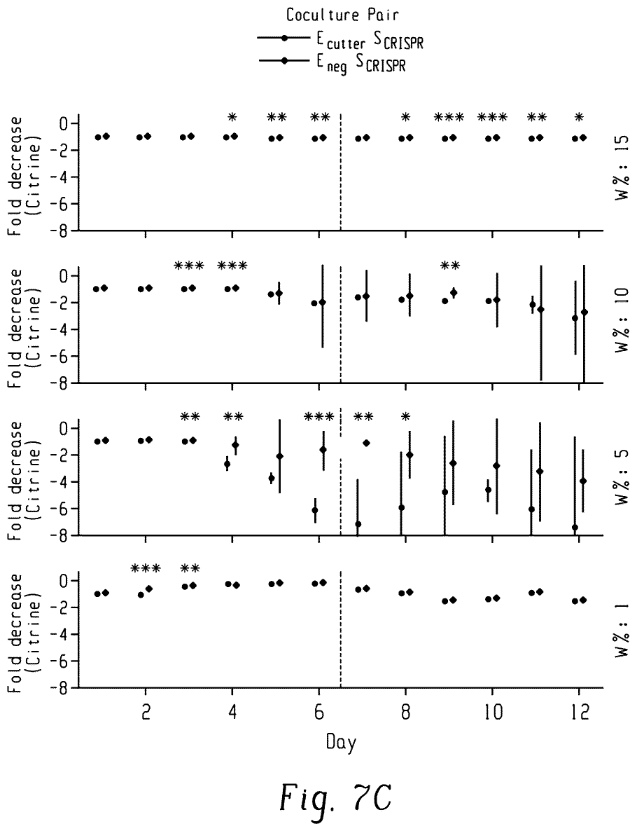

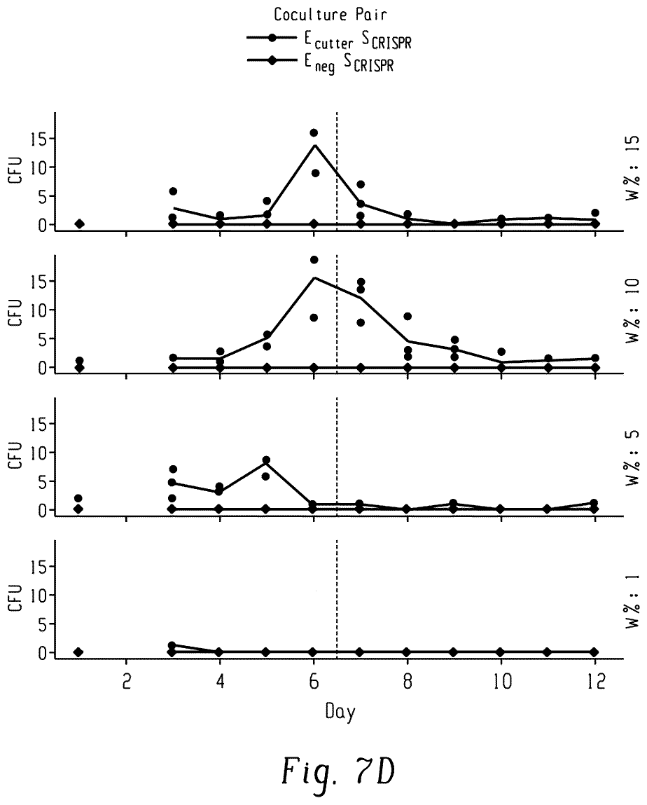

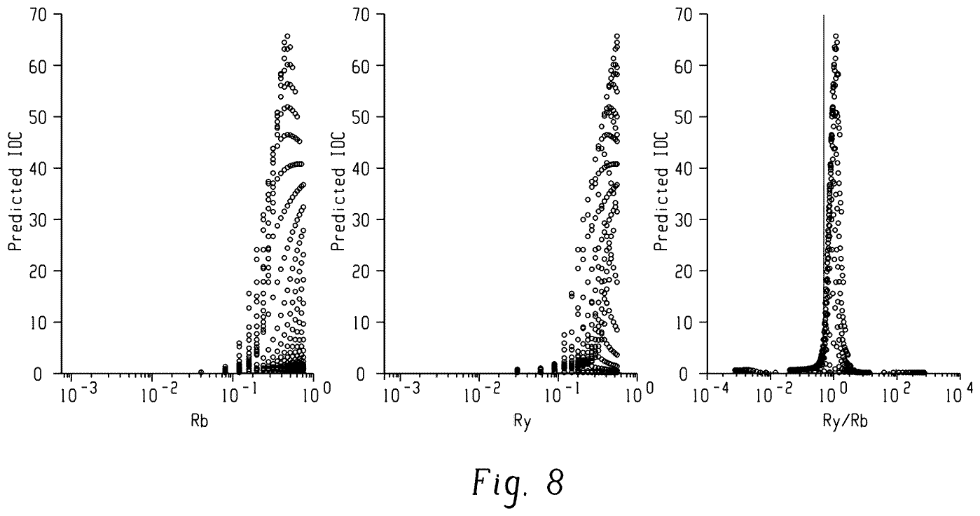

Plates were grown in a customized Tecan Fluent® automated plate handling robot, on a BioShake heater-shaker, kept at 30° C. and rotating at 1000 rpm with a 2 mm orbital. In 15 minute intervals, the Fluent® was programmed to transfer each 96-well plate to a connected Tecan Spark® fluorimeter, in which each well was measured for OD600, mCherry (Ex=575 nm, Em=620 nm, 20 nm bandwidth, gain=60), ymCitrine (Ex=500 nm, Em=545 nm, 20 nm bandwidth, gain=60), and, for CRISPR experiment (FIG. 7), yeBFP (Ex=381 nm, Em=445 nm, 20 nm bandwidth, gain=60). After each plate was measured, it was returned to the BioShake, where it grew for another 15 minutes until the next read. Each plate was grown in this way for roughly 18-24 hr., at which time plates were briefly spun (1 min at 1000×G) to remove droplets from plate seal. Each plate was then diluted 1:10 in fresh media (180 μL media+20 μL previous day's culture) for that day's growth, with another 20 μL diluted into a plate of PBS+0.1% Tween® for flow cytometry (see below). Tecan data was consolidated in Excel format and imported into MATLAB via a custom script, which parses the Tecan Excel export format based on number of plates and channels measured. All further analyses were performed in MATLAB, including normalization, in which all fluorescence measurements were divided by the max reading of that channel: these normalized reads were used for D:R ratios in FIG. 3.

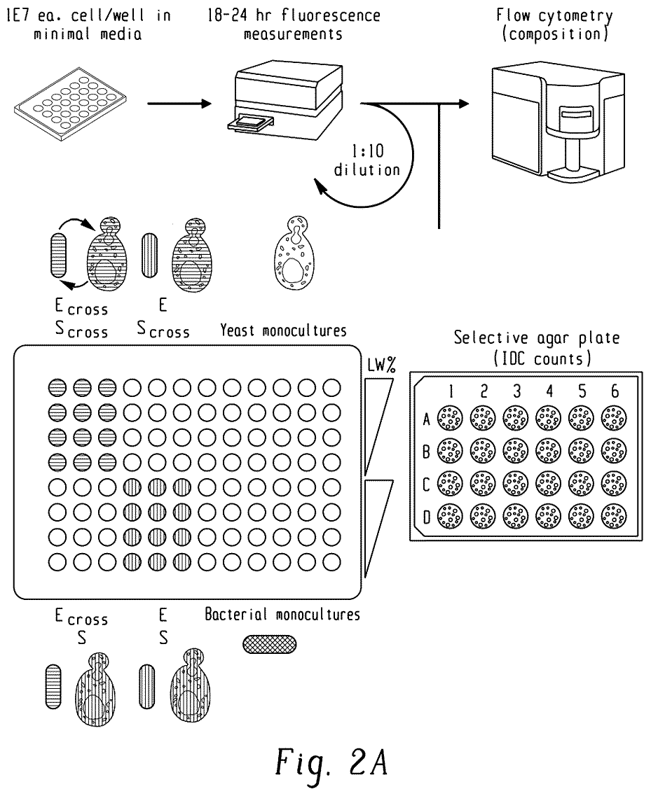

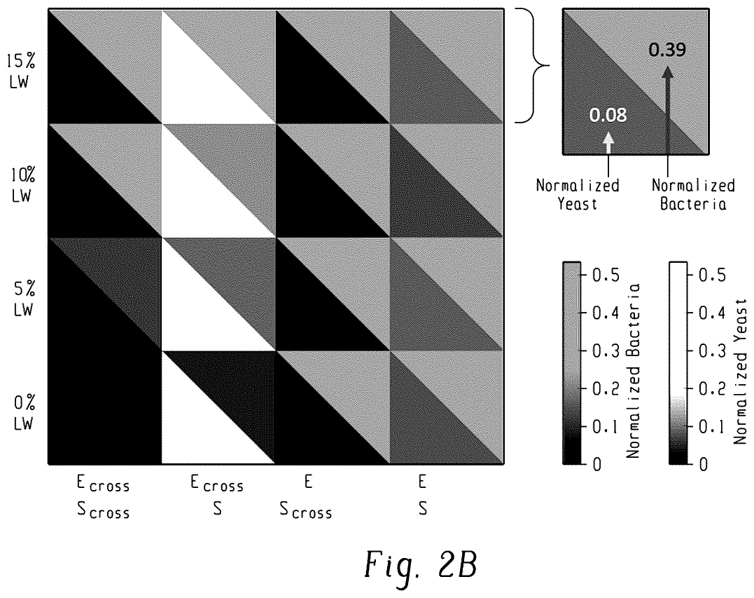

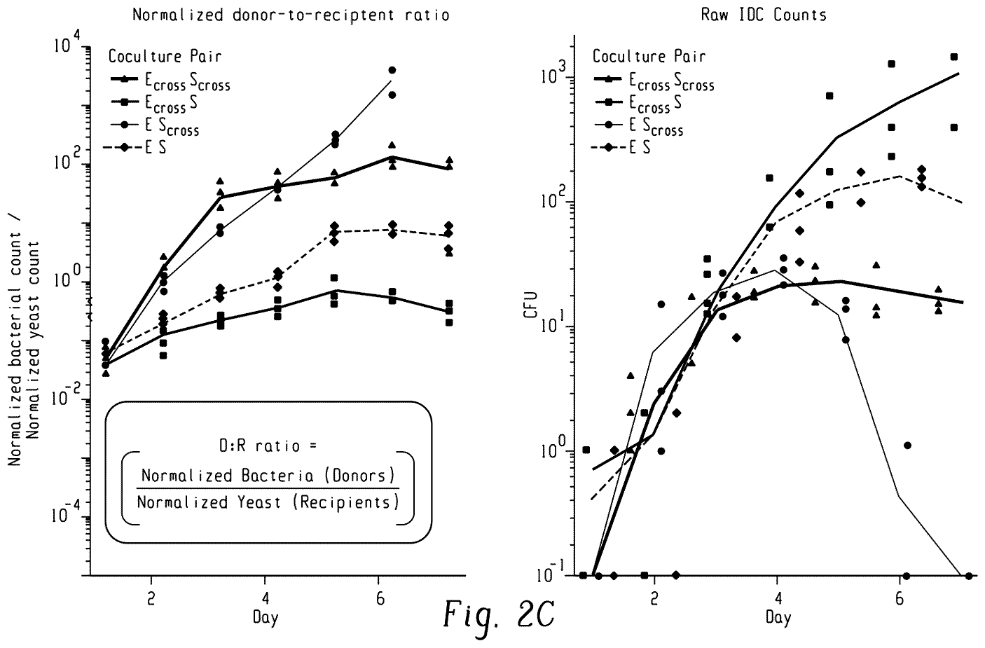

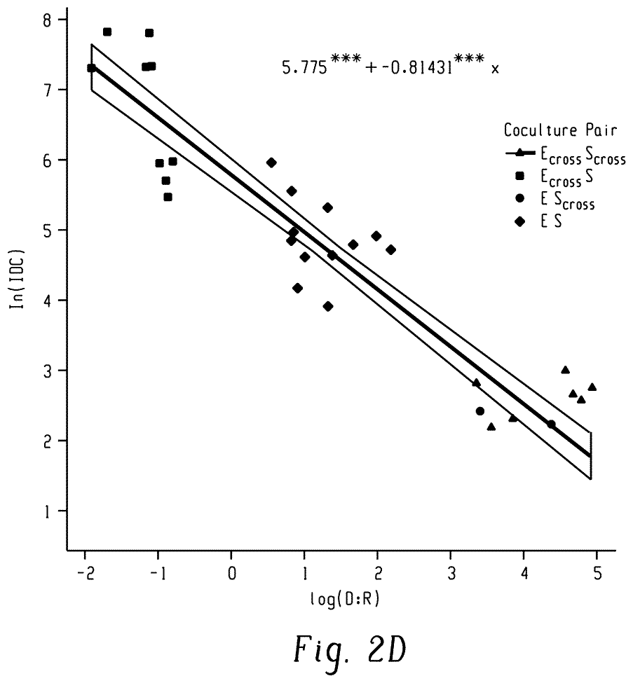

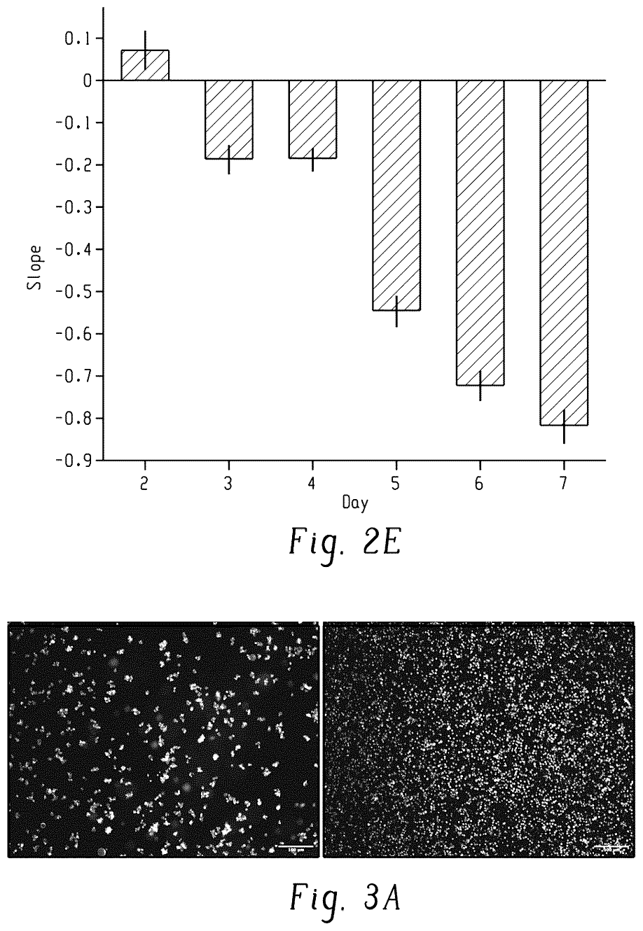

An additional 100 μL of each day's culture was added, undiluted, to a 24-well plate containing IDC-selective SC with 2% agar: for cis-transfer experiments (FIGS. 2, 5, and 6), SC-UH was used, whereas SC-H was used for trans-transfer experiments (FIGS. 3, 5, and 7). IDC plates were then placed in a culture shaker at 30° C. for ˜40) minutes, without lids, to dry. Once dried, IDC plates were incubated for ˜3 days to grow countable transconjugant colonies. Individual transconjugant colonies were counted for CFU, unless wells were saturated, for which estimates were generated based on density relative to countable wells, up to 500. For rescue assay, due to higher counts, cultures after day 2 were serial-diluted up to 1:10,000, in increments of 10× dilutions, and frogged onto SC-UH 2% agar in a 245 mm BioAssay Dish (Corning cat #431111). Countable microcolonies from frogging dilutions were averaged, based on dilution value; thus, saturated microcolonies were ignored.

Colony experiments: Each strain was grown, measured, washed, and diluted as in batch culture experiments. Because 2 μL mixed culture droplets were to seed each colony, the input cell counts were lowered to 1E6 of each cell type. Strains were combined accordingly, then aliquoted into strip tubes, from which we were able to multichannel-pipette ≥18 identical 2 μL mixed colonies onto 2% agar minimal media plates. Each 60 mm plate (Eppendorf cat #0030701011) contained 75:25 SC: M9 with appropriate bacterial antibiotics for plasmid maintenance, 2% agar, and one concentration of amino acids, such that each plate represented a single experimental condition: molten media was aliquoted to plates in equal (15 mL) portions. Once mixed colonies were added to plates, they were allowed to grow at 30° C. for 6 days. Three representative colonies (by eye) were designated after the first day's growth to be repeatedly imaged over the entire time course, while another three were designated to be imaged that day only, after which they would be scraped, washed, and measured by flow cytometry and IDC-plating. All colonies were numbered, upon being selected, to correlate measurements.

Plates were imaged for fluorescence using a Zeiss AxioZoom V16 dissecting microscope, at UW-Madison's Newcomb Imaging Center. Each cis-donor mixed colony was imaged for mCherry (Zeiss Set 43 BP 545/25, FT 570, BP 605/70, 200 ms exposure) and ymCitrine (Zeiss Set 46 HE, EX BP 500/20, BS FT 515, EM BP 535/30, 600 ms exposure), while trans-donor mixed colonies were additionally measured for yeBFP (Zeiss set 49: G365, FT395, BP445/50, 500 ms exposure). All images were taken at 8× zoom. See SI discussion for more information on image processing and analysis.

After imaging, colonies were manually scraped off plates via micropipette tips and diluted into 1.5 mL tubes containing 1 mL water. Each diluted colony was vortexed for ˜30 s to break up colonies and dilute residual agar, then spun at 3000×G for 5 min. 800 μL of water was removed from each tube, cells were resuspended in the remaining ˜200 μL, 100 μL of which was plated for IDC-selection (see Batch Culture methods) and another 20 μL was aliquoted into 180 μL PBS+0.1% Tween® for flow cytometry (see below). After six days of growth, the colonies (1-3) designated for continual microscopy imaging were processed and measured in this way.

Flow cytometry: After diluting cells from culture or colonies (see above) 1:10 into PBS+0.1% Tween (total volume=200 μL) in 96-well round-bottom plates (Fisher Scientific cat #07-200-760), samples were measured for cellular composition using a ThermoFisher Attune™ N×T V6 Flow Cytometer at UW-Madison's Carbone Cancer Center, which includes a 96-well compatible autosampler. Because the sizes of bacteria and yeast are so different, each co-culture was measured twice, with different forward and side scatter voltages for each cell type (monoculture controls were generally measured using only that species' voltage settings, though at least two of the other species were included for each to get baseline counts). Each well was measured for ymCitrine (488 nm laser, 530/30 503LP filters, off target fluorescent) and mCherry (561 nm laser, 620/15 600LP filters), in addition to scatter, using a draw volume of 20 μL, at a flow rate of 200 μL/min.

FCS files exported from the Attune™ were processed via custom MATLAB tools modified for dual-voltage experiments. Gates were drawn per voltage setting to capture all cells of that species based on fluorescence and forward scatter. FCS files were imported, correlated with sample information, and queried for inclusion in each gate. Summary tables for each cell type were consolidated to combine all readings per experiment, upon which noise floors were calculated based on negative controls per voltage setting. Gate-defined cell counts for each species were subtracted by these baselines and converted to total cells per 100 μL, to compare to IDC counts (see IDC prep in Batch Culture methods). Cell counts were further normalized by dividing by the max count for that experiment and cell type: normalized counts were used to generate D:R ratios (FIGS. 2, 5, 6).

Microscopy of culture aggregates: Batch culture samples were diluted to various degrees (depending on day and sample density) in media lacking glucose and amino acids, but with mannose for samples grown with it, in a CellVis 96-well back-walled optical glass-bottom plates (cat #P96-1-N). Plates were loaded onto the stage of an inverted fluorescence microscope (Nikon TiE), enclosed by an opaque incubation chamber. A custom Nikon JOBS script was written to image each well of a plate in three random locations distal to the well edges, with a two second wait time before each photo to allow cells to settle after moving the stage. All wells were imaged at 10× objective for mCherry (Chroma 96365, Ex=560/40×, Em=630/75 m, 200 ms exposure), ymCitrine (Chroma 96363, Ex=500/20×, Em=535/30 m, 600 ms exposure), and yeBFP (Chroma NC296093, Ex=350/50×, Em=460/50 m, 500 ms exposure).

Data analysis and figures: Unless otherwise specified, all data processing was performed using custom MATLAB scripts. Most data plots were generated with the gramm MATLAB toolbox⁶² and flow diagrams were created with BioRender.com.

### Example 1: Tunable Population Dynamics in Batch Culture Affect IDC Outcomes

To determine if IDC frequencies can be controlled by tuning steady state population growth, strains of E. coli and S. cerevisiae were designed to be obligate mutualists when deprived of nutrients. A previously studied yellow-fluorescent yeast strain that is tryptophan auxotrophic (Trp⁻, Δtrp2) and leucine overproducing (Leu⁺⁺, LEU4^{FBR}) was used to develop a corresponding leucine-auxotrophic, tryptophan-overproducing cross-feeder E. coli that expresses mCherry. After screening for optimal co-culture conditions (data not shown), cells were batch cultured for six days in minimal media with a range of strain-dependent leucine and tryptophan concentrations. Relative growth was measured in 15-minute intervals using a Tecan fluorescence reader, and in most cases, additionally measured at day-ends via flow cytometry (FIG. 2A). In most cases, bacterial and yeast populations failed to establish stable cross-feeding with leucine and tryptophan fully removed from media, and competition between species acted as the primary driver of population outcomes. Cross-feeding bacteria (“E_{cross}”) temporarily survived via on leucine secreted by cross-feeding yeast (“S_{cross}”) before the latter is outcompeted, driving down both populations: in contrast, auxotrophic yeasts did not benefit similarly from Trp-overproducing bacteria. Moreover, auxotrophic bacteria survived from WT yeasts (“S”) at 0% leucine and tryptophan (0% LW), in an apparent commensal relationship, suggesting either a low but significant level of basal leucine secretion from S or sufficient yeast lysate for E_{cross} survival. WT bacteria (“E”) did not provide a similar benefit for cross-feeding yeasts (FIG. 2B, data not shown).

IDC between bacteria and yeasts was quantitated by plating 100 μL of each batch culture condition onto IDC-selective media at ˜24-hour intervals. Population effects on IDC were measured both in cis—with the self-transferring plasmid pTA-Mob 2.0—and in trans, via a two-plasmid system including the ori^{T}-lacking pTA-Mob 1.0 and a separate, yeast-selectable transfer plasmid (FIG. 1). As in previous work, markedly lower trans IDC counts were found relative to cis IDC (data not shown). Contrary to previous work demonstrating higher IDC counts with more donor bacteria, an inverse correlation between donor-to-recipient ratios and IDC counts (FIG. 2C), especially for cis IDC (data not shown). This trend became more pronounced with time and manifested as a linear fit on a log-log plot (FIG. 2D). Importantly, the trend was not exclusively due to changes in recipient yeast populations, since the IDC-per-recipient frequency also increased over this time. These findings suggest that, despite the lack of stable cross-feeding at 0% LW, IDC can be controlled by tuning populations, since steady-state ratios of donors-to-recipients are inversely correlated to IDC counts.

### Example 2: Mannoprotein-Based Cell Adhesion Mediates IDC

Since IDC depends on cell-cell collisions in culture, the effect of known adherence mechanisms between E. coli and S. cerevisiae on IDC was explored. Mannoproteins are ubiquitous in fungal cell walls, and type I fimbriae in E. coli bind to these proteins, forming bacteria-yeast “clumps” that can affect cross-feeding dynamics. The batch culture experiments were repeated for population dynamics and IDC with- and without mannose added to the media, which saturates bacterial mannose receptors and reduces clumping. These cultures were measured for fluorescence as per previous experiments, but were also imaged via fluorescence microscopy to measure the extent of clumping.

Fluorescence microscopy analysis replicated previous findings showing that adding mannose to growth media prevented most bacteria-yeast clumping (FIG. 3A). Image analysis demonstrated that the size of yeast clumps—a proxy for number of yeast cells per clump—increased concurrent with the number of bacteria in a clump (“coincident bacteria”), implying that bacteria mediate cell clump formation (data not shown). Interestingly, we found that mannose-infused media prevented nearly all IDC, with only a few samples yielding single-digit IDC counts by the end of a six-day time course, roughly 10 fold fewer than corresponding samples without mannose (FIG. 3C). Moreover, mannose-supplemented samples showed fundamentally altered dynamics for E_{cross}-S pairings, with auxotrophic E_{cross} cells unable to survive at 0% leucine, and with much lower growth at higher percentages of leucine relative to mannose-free samples (FIG. 3D, data not shown). Thus, mannose interrupted the commensal dynamics previously seen without mannose.

### Example 3: Deterministic Models Reveal Limitations of Key Parameters

In order to explore how the “knobs” of the system could be tuned to best affect population ratios and IDC, and to better understand the differences between clumping and non-clumping populations, a set of ordinary differential equations (ODEs) was used to deterministically model our experimental conditions, based on previous work modeling cross-feeding co-culture. The results from mannose-supplemented experiments were first fit to a system of two ODEs representing total bacteria and total yeast (including transconjugants). Latin Hypercube Sampling was run iteratively to randomly sample all parameters within a predicted range and total error between model outcomes and fluorescence data were calculated for bacteria and yeast. This error was used to rank model parameters, which was adjusted and rerun until key results were demonstrated for each cell pairing: namely, susceptibility to amino acid supplementation, steady-state survival, and approximate donor-to-recipient ratio (data not shown).

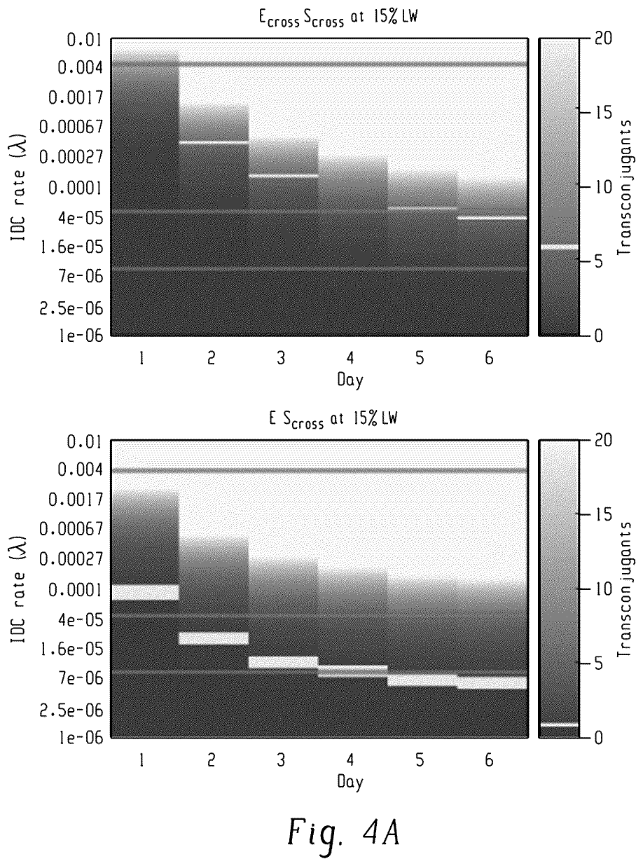

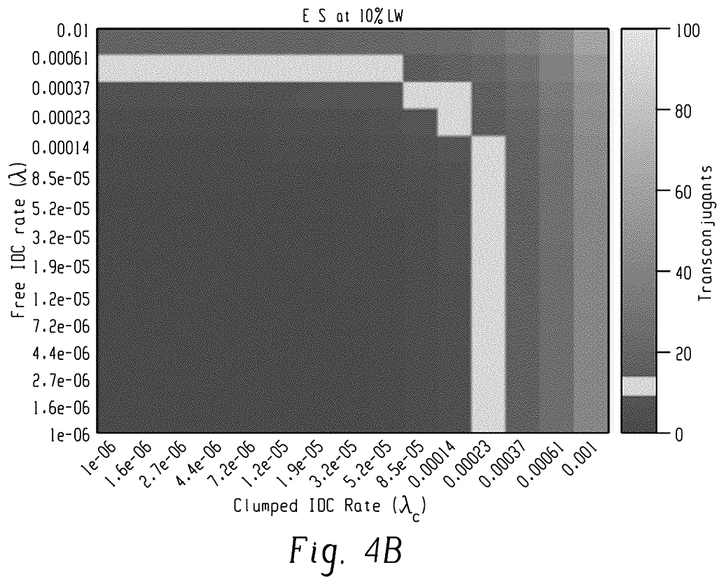

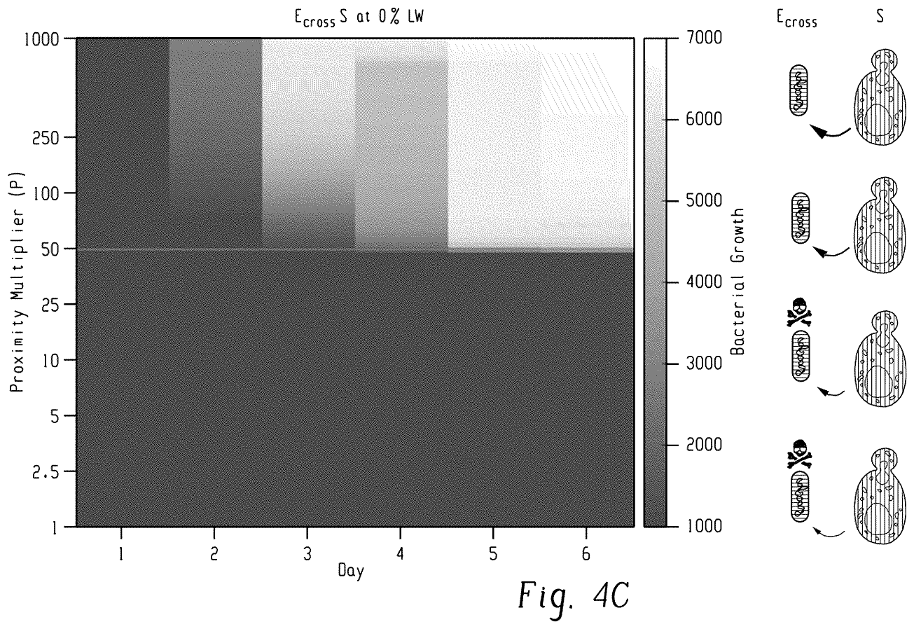

Once a best-fit approximations of parameters in bacterial and yeast growth equations was identified, a wide range of IDC transfer terms (2) were tested against data from mannose-supplemented experiments, to determine order of magnitude for IDC given random cell collisions in culture. Though this plasmid transfer term has been previously approximated at 4*10⁻³ using a similar model for enteric bovine E. coli, in these examples y would have to be significantly lower, roughly between 7*10⁻⁶ and 4*10⁻⁵, to recapitulate our results in media containing mannose (FIG. 4A, data not shown).

Another round of parameterization was performed against measurements of clumped cells growing in mannose-free media, using ODEs modified to include clumping. In this model, IDC was represented by two different transfer terms: γ for free-cell collisions, as per previous fits, and γ_{c} for clumped cells, and. The model fits (data not shown) yielded two possibilities that recapitulated the data: low γ_{c} with γ in the range of 5*10⁻⁴-1*10⁻³—higher than y values found in the free-cell model, thus probably not representative—or low γ with γ_{c} in the range of 2*10⁻⁵-4*10⁻⁵ (FIG. 4B, v). Additionally, a proximity term P was used in this model to account for changes in benefit arising from the proximity of clumped cells, which allows for E_{cross} survival with S. While P mathematically serves to multiply the amino acid secretion term in the model, it could just as likely result from leucine in yeast cell lysate or some other mechanism of bacterial benefit. P value sweeps show an apparent amino-acid secretion increase on the order of 50× from S, to allow E_{cross} cells to grow in 0% leucine (FIG. 4C).

### Example 4: IDC in Colonies Follows Feeding Trends, with Greater Variability

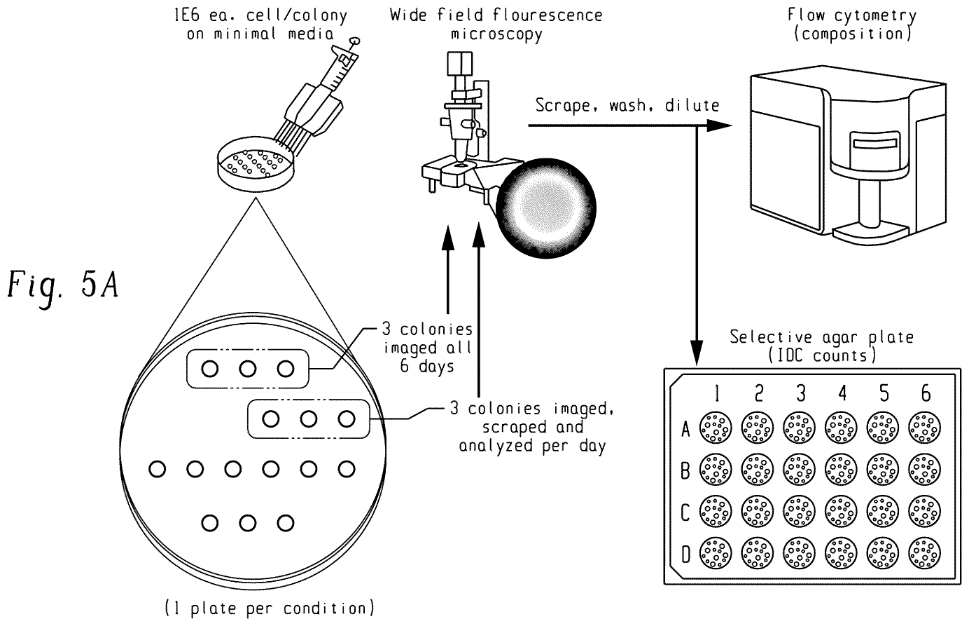

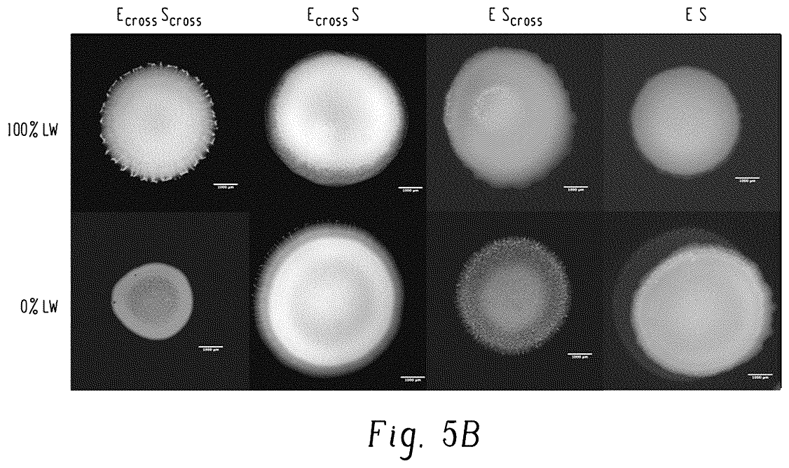

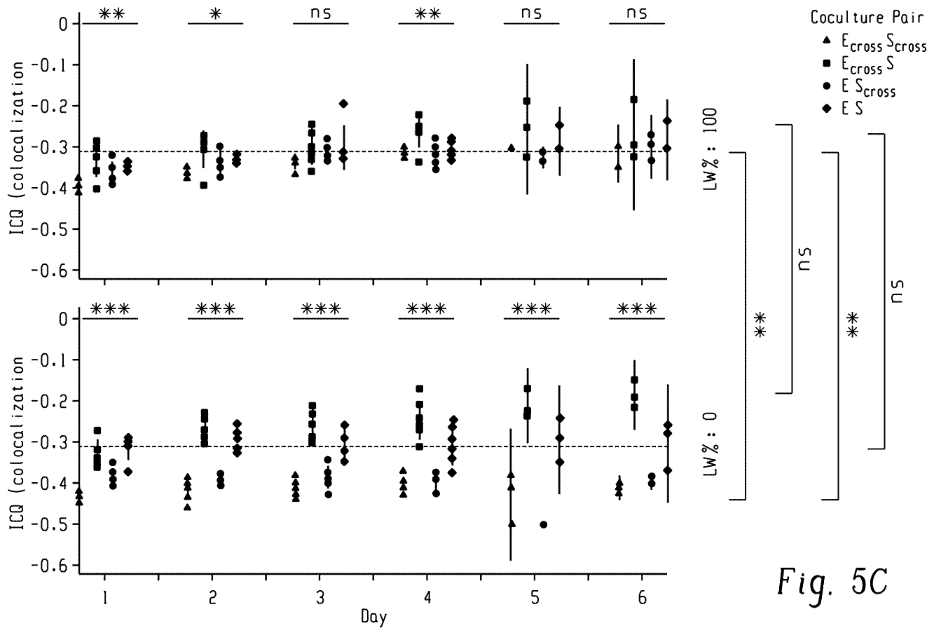

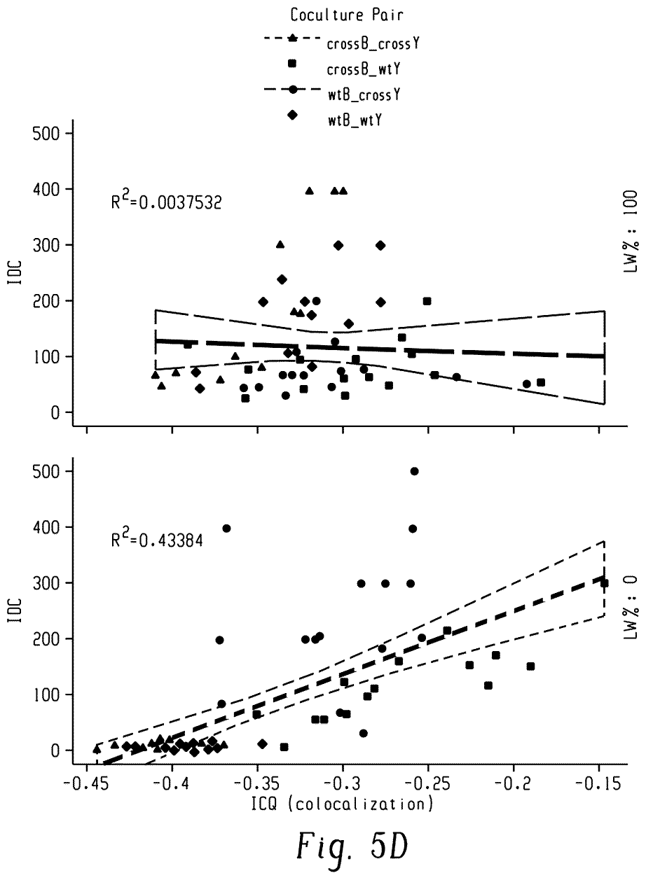

After characterizing IDC in well mixed liquid co-cultures, the next goal was to understand how population dynamics affect IDC in spatially constrained settings, to better predict IDC functionality in natural settings such as biofilms. This experiment emulated “expansion” assays, which have previously demonstrated greater intermixing of mutualistic populations by repeating batch culture initial conditions on 2% agar minimal media plates, except with 10-fold fewer initial cells. ≥18 2 μL mixed-cell droplets were pipetted onto plates and allowed to grow continuously for six days. Three colonies were imaged for 2D spatial distribution each day via wide-field fluorescence microscopy, and another three were then scraped, washed, and diluted for composition and IDC measurements (FIG. 5A,B).

As with batch cultures, there was an inverse correlation between donor-recipient ratios and IDC in most cases, though with greater noise (data not shown). However, IDC-per-recipient frequencies remained relatively constant, unlike cultures (data not shown). These differences from culture conditions might be due to “jackpot” populations, in which a genetic island of transconjugants finds a spatial niche among the stochastic colony front, resulting in a wider range of IDC counts for each condition. Because conjugation has been shown to occur along population boundaries, relative population mixing was determined by calculating colocalization of bacterial and yeast fluorescence signals. While colocalization did positively correlate with overall IDC values, most mixed colonies had very low colocalization, suggesting once more that competition dominates population dynamics between these species (FIG. 5D).

### Example 5: Harnessing Population Dynamics to Rescue a Recipient Population Through IDC

To test whether population-control of IDC can be used to alter a recipient, the next step was to “rescue” starved yeast cells with poor or non-existent growth, via genes carried on the transferred DNA. This was tested with the cis-IDC plasmid pTA-Mob 2.0, which carries HIS3 and URA3 and allows transconjugants to grow in media deficient for uracil and histidine IDC from WT donors mostly failed to rescue U or H-auxotrophic yeast recipients growing in low concentrations of uracil and histidine (% UH), as the bacteria competed the yeasts to collapse before sufficient transconjugant growth could establish (data not shown).

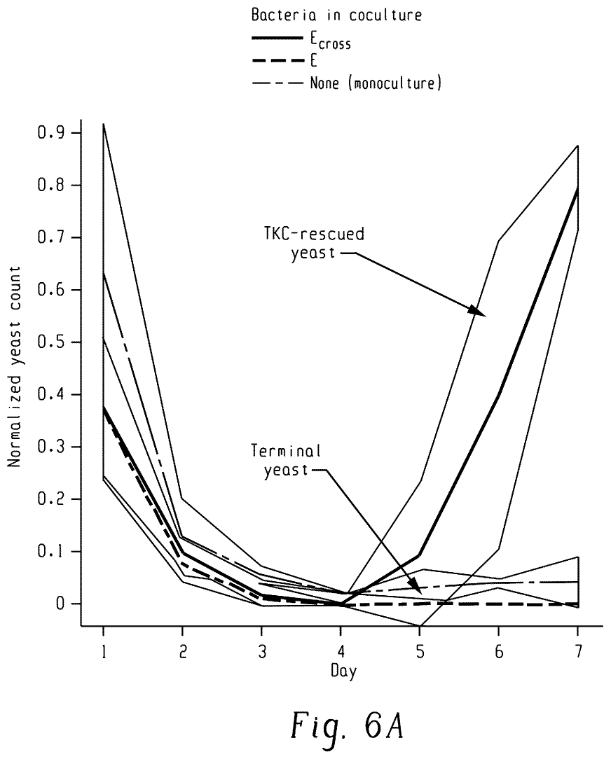

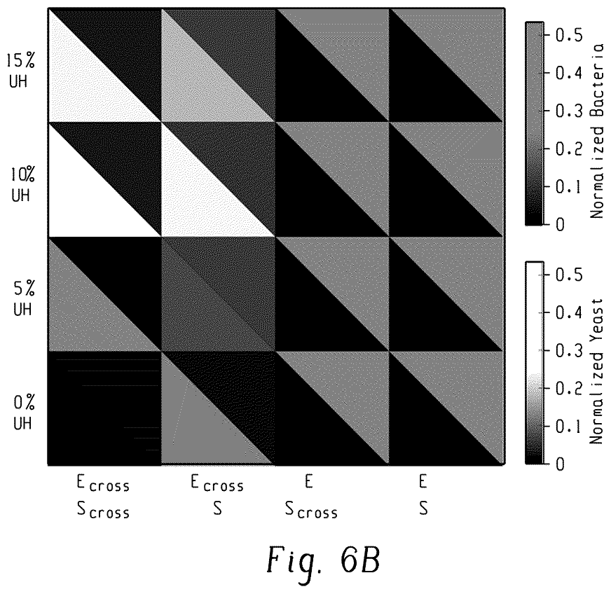

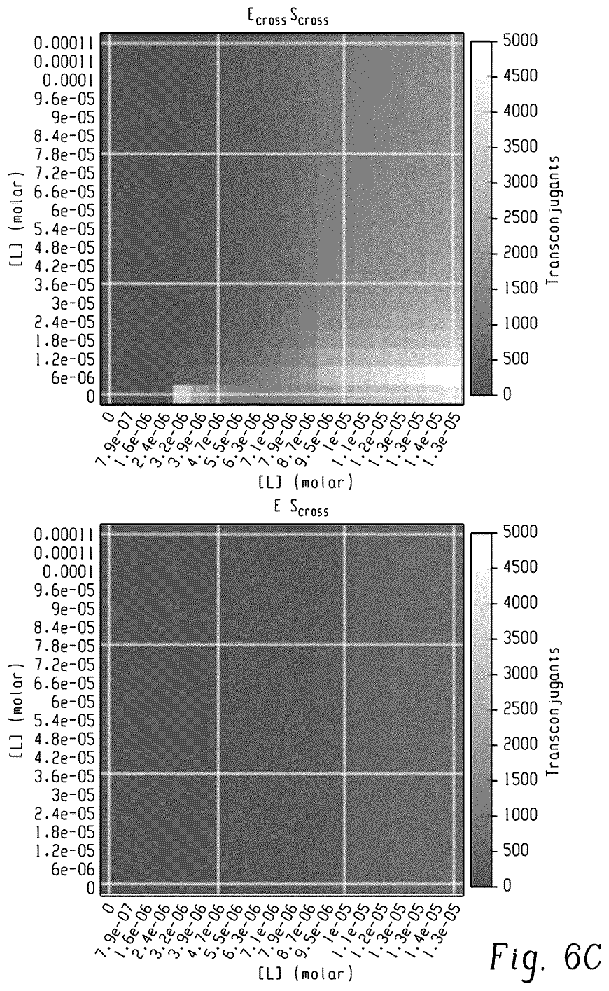

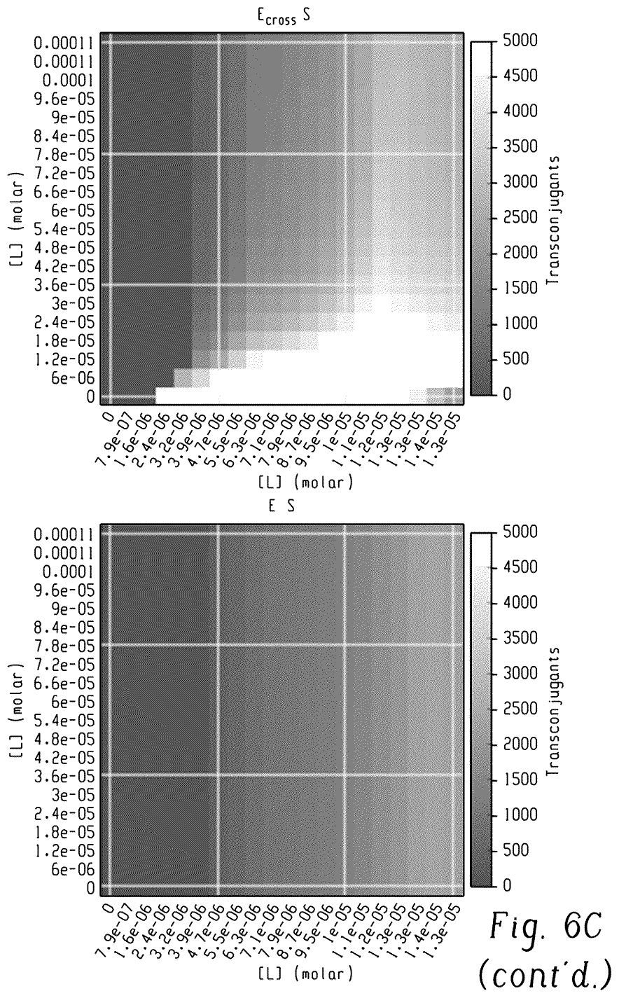

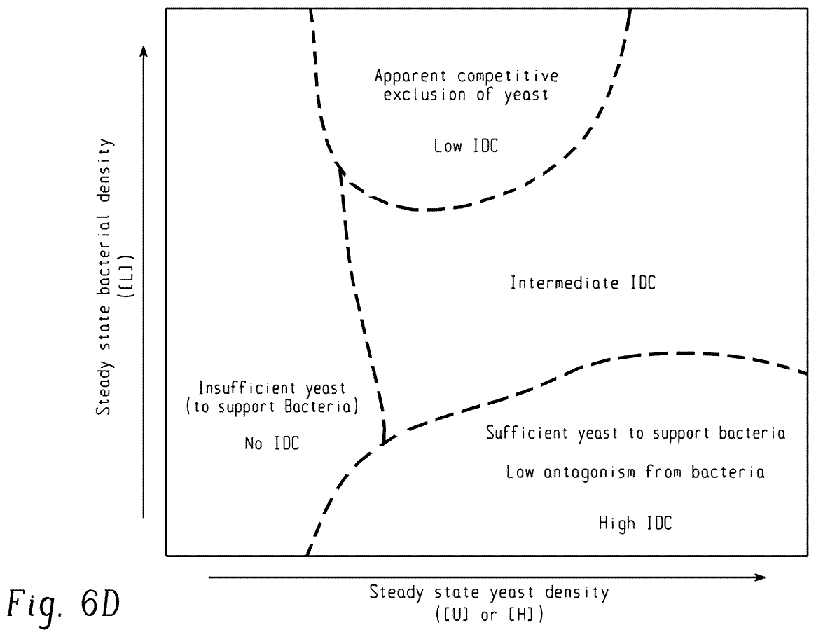

Previous results showed higher IDC for lower donor-to-recipient ratios, so to increase the likelihood of rescue, auxotrophic bacterial donors at 0% leucine were used. These donors can thus only survive if the paired yeasts metabolically support them. Remarkably, a drastic increase in IDC-rescue was observed from E_{cross} donors, for both S_{cross} and S recipients, though this effect varied by uracil and histidine amounts (FIG. 6A,B). E_{cross} rescued both recipient strains with greater speed and efficiency than E did in all cases, though at 0% UH, paired cross-feeder populations collapsed (FIG. 6B). At intermediate concentrations of uracil and histidine—especially 5% UH—rescue showed high stochasticity, as some biological replicates were fully rescued while others collapsed (data not shown). The clumping model was used to predict the range of possible rescue outcomes for each cell pairing over a range of amino acid concentrations. With minimal alterations to account for experimental differences, the model recapitulated the experimental results: for most concentrations of U and H, and with [L] kept low, bacterial competition is minimized, and the greater IDC is possible, allowing for the increased rescue of yeast seen in these experiments (FIG. 6B,C).

### Example 6: IDC-Mediated CRISPR Killing can be Interrupted by Mannose Addition

It was next tested whether IDC-mediated killing could collapse or depress a recipient yeast population. A conjugatable CRISPR/Cas9 system was defined that can be transferred from bacteria to yeast, where it targets a blue fluorescent, URA3-carrying plasmid in recipients, such that destruction of this plasmid would render recipient cells unable to grow in uracil deficient media. Unlike most Cas9 editing systems, which utilize a repair sequence to for homology-directed repair of the cut DNA, this system relied on repeated cutting of the target DNA with no repair, since the goal was simply to suppress the growth of the target cells. Targeting an episomal sequence was also essential, to discern both IDC frequencies and cutting efficiency separately, without the lethality of cutting genomic DNA in recipient yeast (FIG. 7A, data not shown). After verifying that the IDC-Cas9 plasmid is efficient for cutting its target via both direct yeast transformation and IDC (data not shown), cultured cross-feeding yeast (W auxotrophs) containing the BFP-URA3 plasmid were batched at low levels of tryptophan and 0% uracil, along with donor cells that either contained a functional IDC-Cas9 system, or one lacking an ori^{T} sequence and thus unable to transfer DNA. At 1% W, all yeast cultures died out, while at higher levels of W (5% and 10%), competition resulted in depressed yeast levels relative to monoculture yeast growth (FIG. 7B, data not shown). Donors carrying IDC-Cas9 (“cutters”) significantly depressed yeast growth beyond competition-mediated decreases, especially at 5% W, where yeast growth was decreased several-fold beyond non-transferring control donors (FIG. 7C).

To gauge whether any effects of IDC could be reversed by interrupting cell clumps, batch cultures were switched to mannose-supplemented media after six days of growth and allowed to grow for another six days (v). In both co-culture pairings, subsequent yeast growth stopped declining after the media switch and persisted at steady-state levels from day six, ending trends of decline in both co-culture pairings, though never recovering recipients completely to previous (higher) levels (FIG. 7B, data not shown). From this data, the extent to which recipient populations are depressed by competition from co-culture with bacteria, versus IDC-mediated cutting, were discerned since the positive and negative donors are equivalent for fitness and ability to adhere and form pili to recipients (FIG. 7C, data not shown). IDC counts showed reversibility with mannose addition, with IDC dropping after day 6 (FIG. 7D). Since transconjugants are terminal in 0% U, it should be noted that IDC counts are effectively transient “snapshots” of yeasts carrying IDC-Cas9 that have not yet been diluted out of batch-culture or died from starvation.

### Discussion

The experiments described herein build upon advances in synthetic microbiology that allow tunable control of microbial consortia and demonstrate the capacity to significantly alter recipient populations through contact dependent IDC. Importantly, much of the literature regarding IDC considers transfer as a percentage of recipient cells. For many potential applications, however, the per-donor IDC frequency may be equally relevant, especially if donors are utilized as a temporary probiotic. While the would-be cross-feeding mutations did not support steady-state obligate mutualism, auxotrophic E. coli were able to form commensal relationships with even WT S. cerevisiae, yielding some of the highest IDC values we observed. This presents an opportunity to implement conjugation systems in which only donors are engineered.

Given the ubiquity of mannoproteins among fungal cells, and the discrepancy in growth rates between prokaryotic and eukaryotic cells—which allow bacteria to adapt quickly and persist in adverse conditions—it is expected that similar results are achievable with other yeasts, including pathogenic fungi such as Candida glabrata. Malassezia restricta, and Aspergillus fumigatus, for which treatments are limited and in great demand.

Finally, we demonstrated that our insights relating population dynamics to IDC can be applied to functionally alter recipient yeast populations. We “rescued” a low-growing population via IDC transfer of an essential gene, by depressing donor growth and making it dependent on recipient cells, in keeping with our dynamics findings. We also “killed” recipient cells via IDC-mediated Cas9 cutting of an essential gene. In this latter case, we deliberately added a layer of complexity unnecessary to the aim of killing cells, in that we designed yeast recipients to carry the essential gene URA3 episomally, so as to verify the cutting efficiency. Any work aiming only to depress recipient populations could just as easily target the genome, and repeatedly introduce blunt-end cut sites with no available homology-directed repair sequence, simultaneously disrupting the genome and draining cell resources in non-homologous end joining (NHEJ). Perhaps more interesting for future work, however, would be to target more complex functions in recipient cells, such as modifying metabolic pathways in a consortia producing a useful product, or disrupting quorum sensing function in virulent cells, for example, by targeting the farnesol pathway in C. glabrata.

### Full “Free” Clumping Growth Model

Growth equations for bacterial and yeast growth were derived from Pearl-Verhulst logistic growth, for which cells' growth is determined by its (monoculture) growth rate R, carrying capacity K, and death rate D. Modifications for co-culture conditions include deviations from monoculture carrying capacity term and amino acid secretion terms. For the effect of one species on limiting the carrying capacity of the other, a multiplier e is used to account for incomplete ecological niche overlap. The global concentration of amino acid supplemented to media G and amino acid secretion «-dependent on secreting cell's concentration-together modify the growth rate of each species. Monod term k determines a strain's susceptibility to amino acid changes.

Transconjugants grow similarly to yeast, and thus have a nearly identical growth equation. Transconjugants are also added to the system by bacterial and yeast collisions, as modified by IDC transfer term γ. Note that because the growth equation for yeast dY dt is compared to yeast fluorescence data (see below), Y is a representation of all yeast, including transconjugants, and thus has no term depleting cells proportionate to IDC transfer term γ, as is occasionally seen in other equation.

$$\frac{dB}{dt} = {R}_{b} * B ( \frac{{α}_{b} Y + {G}_{b}}{\text{?} Y + \text{?} + \text{?}} ) ( 1 - \frac{B}{\text{?}} - \frac{{c}_{y} Y}{{K}_{y}} ) - {D}_{b} ( \frac{B}{\text{?}} )$$

$$\frac{dY}{dt} = {R}_{y} * Y ( \frac{{α}_{y} B + {G}_{y}}{\text{?} B \vert {G}_{y} \vert {k}_{y}} ) ( 1 - \frac{Y}{{K}_{y}} - \frac{\text{?} Y}{\text{?}} ) - {D}_{y} ( \frac{\text{?}}{{K}_{y}} )$$

$$\frac{dT}{dt} = \text{?} * T ( \frac{{α}_{y} B + \text{?}}{\text{?} B + {G}_{y} + {k}_{y}} ) ( 1 - \frac{T}{{K}_{y}} - \frac{\text{?} Y}{\text{?}} ) - {D}_{y} ( \frac{T}{{K}_{y}} ) + \text{?} ( \frac{B * Y}{B + Y} )$$

$$\text{?} \text{indicates text missing or illegible when filed}$$

Estimates of R and K were first estimated by fitting integrated versions of simplified monoculture growth equations (lacking amino acid terms, death rates, and co-culture modifications) to monoculture fluorescence data at 100% amino acids in solution. Global amino acid concentrations G are known, and initial guesses for cell secretion α are based on literature values for similar strain mutants—0.022 g_{Trp}/CDW*hr for Δtrp^{R} bacteria, and 7*10⁵ molecules_{Leu}/cell*s for Leu⁺⁺ yeasts¹—taken for 1 hour, with an assumed bacterial CDW=3*10⁻¹³ g/cell. Using these, Monod terms k were arithmetically derived for cross-feeder monoculture fits using the equation Rₘₐₓ=R*[AA]/([AA]+k), where [AA]=limited amino acid concentration, over a range of supplemented values.

For all subsequent terms and fits, we used Latin Hypercube Sampling (LHS) to fit a MATLAB ODE solver. We set upper and lower bounds for each parameter, between which 100,000 random parameter guesses were generated, each of which was put through the ODE solver. Each model output, determined by that guess's randomized parameters, was compared to fluorescence data, error between the two was calculated, and random model guesses were ranked by lowest calculated error.

Niche overlap terms c were then estimated using fluorescence of WT co-cultures at 100% amino acids, for each WT donor variant (plasmids carried). Original parameter ranges were set widely for simplified conditions—e.g. c was initially sampled between-1 and 2 for WT pairings (FIG. S8)—then tightened for full-model fitting.

For batch co-culture fitting, fluorescence values were compared to a modified version of ODE solver, in which each days' model outputs were fed into the next day's initial conditions. In this way, we were able to emulate batch culture dilutions at the times experimentally performed and keep the ODE solver in time units of hours. Day 1 growth was ignored for both measurements and model, as variation in cell counts as strains initially adapted to batch culture conditions proved extremely unpredictable and resulted in errant model fits. To allow for experimental variation, noise was incorporated into day-end outputs at a rate of +/−20%, before being fed back into the solver as next-day initial conditions. Initially, error between model and measured data was only calculated for bacterial and yeast growth signals (not IDC values), and fits were ranked according to those errors. Means of each model's 1000 best fits (top 1%) were manually assessed for each cell pairing based on three criteria: 1) susceptibility of each strain to changes in amino acid concentration, 2) steady-state persistence of each strain, and 3) approximate D:R ratio of cells. Means were manually modified to best reflect these priorities, and then fed back into the solver. This process was repeated several times, until model outputs no longer improved upon experimental matches. Model thus represents a local minimum for fitting parameters and is not meant to represent a unique fit.

Once a representative parameter set was acquired for each species growth in co-culture, all parameters besides IDC transfer term γ were fixed, and γ was tested across a range of possible outcomes (10⁻⁶-10⁻²) for each cell pairing and concentration of added leucine and tryptophan. Note that, unlike bacterial and yeast growth equations, each of which maintains a single fluorescence unit (mCherry or ymCitrine), transconjugant equations include a combination of each fluorescence type. To account for this, the transconjugant model converts each cell type from fluorescence to cell count before multiplying by γ—using conversion factors derived by comparing flow data (cell counts) to fluorescence data—after which the entire frequency term is multiplied again by ymCitrine/cell, to maintain units of ymCitrine for transconjugants. Because transconjugants were modeled in units of ymCitrine, and because half of each growth well's volume was plated for IDC counts, model outputs for IDC were divided by 2*Citrine/cell. Averages from each condition's IDC data were incorporated into IDC-sweep plots by altering heatmap colormaps according to where color (row-fraction of total output range) matched IDC counts.

TABLE 3
Free-cell model parameters

| | | | Model Fits per Cell Pairing | | | |
|---|---|---|---|---|---|---|
| Var | Parameter | Unit | E_{cross}-S_{cross} | E_{cross}-S | E-S_{cross} | E-S |
| R_{b} | Bacterial growth rate | hr⁻¹ | 0.75 | 0.66 | 0.75 | 0.75 |
| R_{y} | Yeast growth rate | hr⁻¹ | 0.60 | 0.59 | 0.58 | 0.58 |
| K_{b} | Bacterial carrying capacity | mCherry | 3500 | 2987 | 3201 | 3500 |
| K_{y} | Yeast carrying capacity | ymCitrine | 2600 | 3044 | 3039 | 2998 |
| c_{b} | Ecological niche overlap | Unitless | 0.80 | 0.84 | 0.80 | 0.80 |
| | (effect of bacteria on yeast) | | | | | |
| c_{y} | Ecological niche overlap | Unitless | 0.95 | 0.99 | 0.90 | 0.90 |
| | (effect of yeast on bacteria) | | | | | |
| G_{b} | Global amino acid (for | Molar | 100% leucine = 7.622*10⁻⁴ M | | | |
| | dependent bacteria) | | | | | |
| G_{y} | Global amino acid (for | Molar | 100% tryptophan = 2.449*10⁻⁴ M | | | |
| | dependent yeast) | | | | | |
| α_{b} | Secreted amino acid (for | Molar/ | 1E−4 | 1E−10 | 1E−4 | 1E−10 |
| | dependent bacteria) | mCitrine | | | | |
| α_{y} | Secreted amino acid (for | Molar/ | 1E−9 | 1E−9 | 1E−12 | 1E−12 |
| | dependent yeast) | mCherry | | | | |
| k_{b} | Monod term for dependent | Molar | 2E−6 | 2E−6 | 0 | 0 |
| | bacteria | | | | | |
| k_{y} | Monod term for dependent | Molar | 2.5E−5 | 0 | 2.5E−5 | 0 |
| | yeast | | | | | |
| D_{b} | Bacterial death rate | hr⁻¹ | 0.455 | 0.455 | 0.455 | 0.455 |
| D_{y} | Yeast death rate | hr⁻¹ | 0.50 | 0.547 | 0.570 | 0.498 |
| γ | IDC transfer term | Unitless | 4E−5 | Unknown | 7E−6 | Unknown |

### Full Clumping Growth Model

After testing the free cell model against experimental data, deterministic equations were modified to capture aggregation (“clumping”) dynamics between the species. Specifically, three equations were added to the system, tracking 1) formation of clumps, as determined by some constant rate of aggregation for every random free-cell collision; 2) total clumped bacteria, based on both growth of already-clumped bacteria, or additional collisions between clumps and free bacteria; 3) total clumped yeast, based on growth of already-clumped yeast or additional collisions between clumps and free yeast. The latter clumped-cell growth equation terms are similar to free-cell growth, except for modified growth rates (starting values ˜⅓ free cell values) and proximity terms P, which allow for altered amino-acid feeding from opposing cell type in a clump. Free-cell growth equations were additionally modified to be carrying capacity-limited by summing clumped and free cells in the total per species. Finally, the transconjugant equation was modified to include a second IDC transfer term γ_{c}, based on total number of clumped-bacteria and clumped-yeast interactions. Growth rate for transconjugants was also changed to the clumped-yeast rate, to reflect the expectation that most transconjugants require clumping at some point.

$$\frac{dB}{dt} = ( {R}_{b} * B ) ( \frac{{α}_{b} Y + {G}_{b}}{\text{?} + {G}_{b} + \text{?}} ) ( 1 - \frac{( B + \text{?} )}{\text{?}} - \frac{{c}_{y} ( Y + \text{?} )}{{K}_{y}} ) - \text{?} ( \frac{B}{\text{?}} )$$

$$\frac{dT}{dt} = \text{?} * T ( \frac{\text{?} B + {G}_{y}}{\text{?} B + {G}_{b} + \text{?}} ) ( 1 - \frac{\text{?} + \text{?}}{\text{?}} - \frac{{c}_{b} ( B + \text{?} )}{\text{?}} ) - \text{?} ( \frac{T}{\text{?}} ) + \text{?} ( \frac{B * Y}{B + Y} ) + \text{?} ( \frac{\text{?}}{\text{?}} )$$

$$\frac{dT}{dt} = \text{?} ( \frac{\text{?}}{\text{?}} )$$

$$\frac{\text{?}}{\text{?}} = ( \text{?} ) ( \frac{\text{?}}{\text{?}} ) ( 1 - \frac{\text{?}}{\text{?}} - \frac{\text{?}}{\text{?}} ) - \text{?} ( \frac{\text{?}}{\text{?}} ) + \text{?} ( \frac{\text{?}}{\text{?}} )$$

$$\frac{\text{?}}{\text{?}} = ( \text{?} ) ( \frac{\text{?}}{\text{?}} ) ( 1 - \frac{\text{?}}{\text{?}} - \frac{\text{?}}{\text{?}} ) - \text{?} ( \frac{\text{?}}{\text{?}} ) + \text{?} ( \frac{\text{?}}{\text{?}} )$$

$$\text{?} \text{indicates text missing or illegible when filed}$$

Clumped-cell dynamics were fit similarly to free-cell equations, using LHS and the ODE batch solver. Model outputs were additionally modified in two ways. Clumped-bacteria outputs were limited to 10 cells per clump (based on image analyses, see discussion above), beyond which clumped bacteria counts were subtracted from clumped-bacteria model outputs for each day and added to free-bacteria counts, before the next day's growth was modeled. Final clumped-cell model outputs were added to total free-cell outputs before comparing to fluorescence data, which doesn't distinguish between the two.

To use image analyses of batch culture clumping as comparative data for the model, dynamic information on number of clumps, total clumped bacteria, and total clumped yeast were acquired by the following means (see Image Analysis for determination of clump numbers and number of cells). The total number of clumps was taken as a fraction of all yeast, based on determination of number of yeast in each clump. Thus, a fractional term of clumps/total yeast could be multiplied by the ymCitrine fluorescence signal to get the number of clumps in units of ymCitrine, regardless of the fraction of cell culture was imaged to determine clump count. The number of clumped yeast was calculated similarly, but tracking clumped yeast per total yeast, and multiplying by total yeast ymCitrine signal. For clumped bacteria, coincident bacteria on all clumped yeast (>2 yeast per event) was taken as a fraction of total clumps, to get clumped bacteria per clumps. Upon deriving number of clumps in terms of ymCitrine, this could be multiplied by clumped bacteria per clumps term to get clumped bacteria in terms of ymCitrine, which was then converted to mCherry by conversions described in the discussion of IDC sweeps for the free-cell model. All three metrics—each only determined at day-ends—were imputed for intermediate times as linear increases from 1/10^{th} the previous day's metric (day 1 assumed=0).

Batch co-culture LHS model outputs were compared similarly to free-cell LHS fitting, using error between the model and experimental data, but here error was calculated between each experimental result: total bacteria, total yeast, IDC counts, number of clumps, clumped bacteria, and clumped yeast. Upon deriving fits that recapitulated main experimental outcomes, key clumping parameters P and γ_{c} were swept across a range of values to find approximate viable values. P value sweeps show an apparent amino-acid secretion increase on the order of 50× from S, for E_{cross} cells to be able to grow in 0% leucine. IDC transfer term sweeps of γ and γ_{c} yielded several values of each that were able to recapitulate the data, though they roughly fell into two categories: low γ_{c} with γ in the range of 3*10⁻⁴-6*10⁻⁴, or low γ with γ_{c} in the range of 2*10⁻⁵-4*10⁻⁵. While this model assumes that the vast majority of transconjugants result from clumped interactions, it's not clear how transient clumps are, so the free-cell IDC transfer term here accounts for IDC from cells measured to be “free” despite having previously been clumped at some time between measurements.

TABLE 4
Clumped-cell model parameters

| | | | Model Fits per Cell Pairing | | | |
|---|---|---|---|---|---|---|
| Var | Parameter | Unit | E_{cross}-S_{cross} | E_{cross}-S | E-S_{cross} | E-S |
| R_{b} | Free bacterial growth rate | hr⁻¹ | 0.75 | 0.75 | 0.75 | 0.75 |
| R_{y} | Free yeast growth rate | hr⁻¹ | 0.58 | 0.58 | 0.58 | 0.58 |
| R_{cb} | Clumped bacterial growth rate | hr⁻¹ | 0.25 | 0.25 | 0.25 | 0.25 |
| R_{cy} | Clumped yeast growth rate | hr⁻¹ | 0.18 | 0.18 | 0.18 | 0.18 |
| R_{c} | Clumping rate | Unitless | 0.03 | 0.05 | 0.03 | 0.05 |
| K_{b} | Bacterial carrying capacity | mCherry | 7566 | 7269 | 5590 | 6790 |
| K_{y} | Yeast carrying capacity | ymCitrine | 2822 | 2700 | 2637 | 2412 |
| c_{b} | Ecological niche overlap | Unitless | 0.80 | 0.69 | 0.90 | 0.70 |
| | (effect of bacteria on yeast) | | | | | |
| c_{y} | Ecological niche overlap | Unitless | 0.91 | 0.95 | 0.90 | 0.93 |
| | (effect of yeast on bacteria) | | | | | |
| G_{b} | Global amino acid (for | Molar | 100% leucine = 7.622*10⁻⁴ M | | | |
| | dependent bacteria) | | | | | |
| G_{y} | Global amino acid (for | Molar | 100% tryptophan = 2.449*10⁻⁴ M | | | |
| | dependent yeast) | | | | | |
| α_{b} | Secreted amino acid (for | Molar/ | 9.2E−5 | 1E−8 | 9.2E−5 | 1E−8 |
| | dependent bacteria) | mCitrine | | | | |
| α_{y} | Secreted amino acid (for | Molar/ | 1E−9 | 1E−9 | 1E−12 | 1E−12 |
| | dependent yeast) | mCherry | | | | |
| P_{b} | Proximity multiplier for α_{b} | Unitless | 50 | 50 | 1 | 1 |
| P_{y} | Proximity multiplier for α_{y} | Unitless | 1 | 1 | 1 | 1 |
| k_{b} | Monod term for dependent | Molar | 2E−6 | 2E−6 | 0 | 0 |
| | bacteria | | | | | |
| k_{y} | Monod term for dependent | Molar | 1.2E−5 | 0 | 1.2E−5 | 0 |
| | yeast | | | | | |
| D_{b} | Bacterial death rate | hr⁻¹ | 0.46 | 0.50 | 0.52 | 0.46 |
| D_{y} | Yeast death rate | hr⁻¹ | 0.46 | 0.39 | 0.45 | 0.48 |

| | | | |
|---|---|---|---|
| γ | Free IDC transfer term | Unitless | ~2E−4-1E−3 (see FIG. 4) |
| γ_{c} | Clumped IDC transfer term | Unitless | ~1E−4-6E−4 (see FIG. 4) |

### Rescue Growth Model

To apply insights from the clumping model, rescue conditions were tested within the clumping model via the following modifications. Here, yeast cells are selected for IDC events, and there's no limitation of tryptophan (required externally by S_{cross}). To account for this experimental change, the entire amino acid term for yeast growth was based upon limitations of uracil and histidine (required externally for both yeast strains, and carried by MOBI plasmid)—a change accounted for in changes to input parameters G—but the entire amino acid term was removed from the transconjugant ODE, making them agnostic to terms G, α, and P. Moreover, because we assume rescued yeast to not stay primarily clumped over a long period, the growth rate for transconjugants was assumed to fall somewhere between that of clumped yeast and free yeast. Finally, unlike previous transconjugant equations, in which transconjugants were primarily carrying-capacity limited by non-transconjugant yeast, here the opposite is likelier true, for any rescue conditions (that would allow yeast numbers to approach carrying capacity), so Tis entered into carrying capacity limitation for free bacteria, free yeast, and transconjugants.

$$\frac{dT}{dt} = ( \frac{\text{?}}{\text{?}} ) * T ( 1 - \frac{\text{?}}{{K}_{y}} - \frac{{c}_{b} B}{\text{?}} ) - {D}_{y} ( \frac{T}{{K}_{y}} ) + \text{?} ( \frac{B * Y}{B + Y} ) + \text{?} ( \frac{\text{?} * \text{?}}{\text{?} + \text{?}} )$$

$$\text{?} \text{indicates text missing or illegible when filed}$$

To model the phase space of transconjugant outcomes for a range of bacterial and yeast fitness, based on limited amino acids, concentrations of leucine (E_{cross}-dependent) and uracil/histidine (all yeast-dependent) G were tested against other fixed parameter outcomes from the clumped-cell model. Because molar concentrations of uracil (S-_{cross}-dependent) and histidine (S-dependent) are similar for 100% KS solution (0.0954 mM histidine, 0.178 mM uracil), both were assumed equal at 100% (0.100 mM). Monod terms k were modified to reflect amino-acid sensitivity differences from cross-fed coculture experiments, with E_{cross} maintaining its value for leucine dependence, E maintaining its lack of sensitivity (k_{b}=0), and setting both yeast strains to the value found for S_{cross} sensitivity to tryptophan (see Table S2), making both S_{cross} and S equally sensitive to U or H.

Concentrations of amino acids were swept over the range of 0-15%, as per many experimental conditions, even though the rescue assay kept [L] at 0% (uracil and histidine ranged from 0% to 15%). Model anomalies arose when setting all amino acids at or near 0, in which IDC values far surpassed possible ranges (>10¹⁰), presumably due to small denominators in cell collision equation terms. To account for this, model outputs for which either bacterial or yeast counts dropped below 1 (after converting from fluorescence, for both clumped- and free-cells) were zeroed out for IDC at those times. This modification had no perceptible changes for amino acids not near 0%.

TABLE 5
Rescue model parameters (those not listed are the same as in Table 4)

| | | | Model Fits per Cell Pairing | | | |
|---|---|---|---|---|---|---|
| Var | Parameter | Unit | E_{cross}-S_{cross} | E_{cross}-S | E-S_{cross} | E-S |
| G_{y} | Global amino acid (for | Molar | 100% Uracil/Histidine = 1.0*10⁻⁴ M | | | |
| | dependent yeast) | | | | | |
| α_{y} | Secreted amino acid (for | Molar/ | 1E−20 | 1E−20 | 1E−20 | 1E−20 |
| | dependent yeast) | mCherry | | | | |

| | | | |
|---|---|---|---|
| γ | Free IDC transfer term | Unitless | 1E−8 |
| γ_{c} | Clumped IDC transfer term | Unitless | 2.5E−4 |

### Simplified Model

The simplified model takes out everything except growth rates (R), ecological niche overlaps (c), carrying capacities (K) and conjugative transfer term (gamma). The simplified model ignores the clumping and feeding effects.

$$Bacterial growth : \frac{dB}{dt} = {R}_{b} * B ( 1 - \frac{B}{\text{?}} - \frac{{c}_{y} Y}{\text{?}} )$$

$$Yeast ( non - conjugated ) growth : \frac{dY}{\text{?} t} = \text{?} * Y ( 1 - \frac{\text{?}}{\text{?}} - \frac{{c}_{b} B}{{K}_{b}} ) - \text{?} ( \frac{B * Y}{\text{?} + \text{?}} )$$

$$Transconjugant ( conjugated yeast ) growth : \frac{dT}{dt} - \text{?} T ( 1 - \frac{T}{\text{?}} - \frac{\text{?} B}{\text{?}} ) + \text{?} ( \frac{B * Y}{B + Y} )$$

$$\text{?} \text{indicates text missing or illegible when filed}$$

R_{b}=growth rate of bacteria
K_{b}=Bacterial carrying capacity
K_{y}=Yeast carrying capacity
B=starting number of bacteria cells
Y=starting number of yeast cells
c_{y}=Ecological niche overlap (effect of bacteria on yeast)
R_{y}=growth rate of yeast
c_{b}=Ecological niche overlap (effect of yeast on bacteria)
T=numbers of transconjugants

The rate terms Ry and Rb were by their resultant IDC (total conjugation), and scatterplots were run looking at how Rb, Ry, and Ry/Rb each relate to IDC. (FIG. 7) On the plot of Ry/Rb (right column), the expected ratio of rates for the strains inherently was used: E. coli grows about twice as fast as S. cerevisiae when each species is grown on its own, so the vertical black line in that plot is set at Ry/Rb=0.5. Assuming errors, Ry/Rb=0.3 is expected to provide the desired results corresponding to higher IDC.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of inter-domain modification of a fungal population, comprising
co-culturing a bacterial population and the fungal population under growth conditions for the bacteria,
wherein the bacterial population comprises a first bacterial plasmid comprising a bacterial selection marker and an operon encoding a type IV secretion system for conjugative transfer, and a second bacterial plasmid comprising a yeast selection marker, an expression cassette for expression of a transferred DNA sequence, and an origin of transfer sequence for conjugative transfer of the second bacterial plasmid; or
wherein the bacterial population comprises a single bacterial plasmid comprising a bacterial selection marker, a yeast selection marker, an expression cassette for expression of a transferred DNA sequence, an origin of transfer sequence for conjugative transfer of the single bacterial plasmid, an operon encoding a type IV secretion system for conjugative transfer, and
maintaining growth of the bacterial population during the co-culturing by controlling an essential nutrient for growth of the bacterial population,
wherein the second bacterial plasmid or the single bacterial plasmid is transferred to at least a portion of the fungal population to provide the inter-domain modification.

2. The method of claim 1, wherein in maintaining growth of the bacterial population during the co-culturing by controlling an essential nutrient for growth of the bacterial population, the yeast growth rate divided by bacterial growth rate is greater than 0.3.

3. The method of claim 1, wherein the yeast growth rate (dY/dt) and the bacterial growth rate (dB/dt) are calculated by:
$$Bacterial growth : \frac{dB}{dt} = {R}_{b} * B ( 1 - \frac{B}{\text{?}} - \frac{\text{?} Y}{\text{?}} )$$
$$Yeast ( non - conjugated ) growth : \frac{dY}{dt} = \text{?} * Y ( 1 - \frac{Y}{\text{?}} - \frac{{c}_{b} B}{\text{?}} ) - \text{?} ( \frac{B * Y}{B + Y} )$$
$$\text{?} \text{indicates text missing or illegible when filed}$$
Wherein
R_{b}=free growth rate of bacteria,
K_{b}=Bacterial carrying capacity,
K_{y}=Yeast carrying capacity,
B=starting number of bacteria cells,
Y=starting number of yeast cells,
C_{y}=Ecological niche overlap (effect of bacteria on yeast),
R_{y}=free growth rate of yeast, and
c_{b}=Ecological niche overlap (effect of yeast on bacteria).

4. The method of claim 1, wherein maintaining growth of the bacterial population during the co-culturing by controlling an essential nutrient for growth of the bacterial population comprises limiting the concentration of the essential nutrient in the co-culture and providing the essential nutrient by overproduction of the essential nutrient from the fungal population, herein the bacterial population is auxotrophic for the essential nutrient.

5. The method of claim 4, wherein the bacterial population overproduces a second essential nutrient, and the fungal population is auxotrophic for the second essential nutrient, and wherein the method comprises limiting the second essential nutrient in the co-culture.

6. The method of claim 1, further comprising adding mannose to the co-culture to reduce a percentage of fungi undergoing inter-domain modification.

7. The method of claim 1, wherein the transferred DNA sequence comprises a gene encoding a metabolic enzyme, a gene encoding a drug resistance marker, or a gene encoding an auxotrophy.

8. The method of claim 1, wherein the bacterial population comprises the first and second bacterial plasmid, wherein the second bacterial plasmid further comprises an expression cassette for a Cas9 nuclease, wherein the transferred DNA sequence expresses a guide RNA, wherein the fungal population comprises a target sequence for the guide RNA, wherein the Cas9 nuclease and the guide RNA cut the target sequence in the fungal population, but no repair sequence is provided.

9. The method of claim 7, wherein the target sequence in the fungal population is in an essential yeast gene, and wherein cutting the target sequence kills the yeast.

10. The method of claim 1, wherein the bacteria is E. coli, and the yeast is Saccharomyces sp, Lachancea sp., Kluyveromyces sp., Pichia sp., Candida sp., Malassezia sp., Aspergillus sp., or Yarrowia sp.

11. The method of claim 1, wherein the bacteria is E. coli, and the yeast is S. cerevisiae, Candida glabrata, Malassezia restricta, or Aspergillus fumigatus.

12. The method of claim 1, wherein co-culturing is in batch or in continuous culture or on a solid substrate.

13. A method of intra-domain killing of a fungal population in an infected host, comprising
administering a bacterial population to the host, wherein the bacterial population comprises a first bacterial plasmid comprising a bacterial selection marker and an operon encoding a type IV secretion system for conjugative transfer, and a second bacterial plasmid comprising a yeast selection marker, an expression cassette for a Cas9 nuclease, an expression cassette for expression of a guide RNA, and an origin of transfer sequence for conjugative transfer of the second bacterial plasmid; wherein no repair sequence for homology-directed repair of the Cas9-mediated double-strand break is provided, and wherein the Cas9 nuclease and the guide RNA cut a target sequence in the fungal population,
wherein the second bacterial plasmid is transferred to at least a portion of the fungal population to provide the intra-domain killing by cutting the target sequence in the fungal population,
or
administering a bacterial population to the host, wherein the bacterial population comprises a single bacterial plasmid comprising a bacterial selection marker, a yeast selection marker, an expression cassette for expression of a transferred DNA sequence, an origin of transfer sequence for conjugative transfer of the single bacterial plasmid, an operon encoding a type IV secretion system for conjugative transfer, and
wherein the single bacterial plasmid is transferred to at least a portion of the fungal population to provide the intra-domain killing by cutting the target sequence in the fungal population.

14. The method of claim 13, wherein cutting the target sequence in the fungal population inactivates an essential gene in the fungal population.

15. The method of claim 14, wherein the essential gene is in a yeast plasmid.

16. The method of claim 13, wherein administering is oral or parenteral administration.

17. A modified bacteria comprising a first bacterial plasmid comprising a bacterial selection marker, and a second bacterial plasmid comprising a yeast selection marker, an expression cassette for a Cas9 nuclease, an expression cassette for expression of a guide RNA, an origin of transfer sequence for conjugative transfer of the second bacterial plasmid, and an operon encoding a type IV secretion system for conjugative transfer; wherein no repair sequence for homology-directed repair of the Cas9-mediated double-strand break is provided, and wherein the Cas9 nuclease and the guide RNA cut a target sequence in a fungal population,
or
a single bacterial plasmid comprising a bacterial selection marker, a yeast selection marker, an expression cassette for expression of a transferred DNA sequence, an origin of transfer sequence for conjugative transfer of the single bacterial plasmid, an operon encoding a type IV secretion system for conjugative transfer, and an expression cassette for a Cas9 nuclease, wherein no repair sequence for homology-directed repair of the Cas9-mediated double-strand break is provided, and wherein the Cas9 nuclease and the guide RNA cut a target sequence in a fungal population.

18. The modified bacteria of claim 17, wherein cutting the target sequence in the fungal population inactivates an essential gene in the fungal population.

19. The modified bacteria of claim 18, wherein the essential gene is in a yeast plasmid.

20. A pharmaceutical composition comprising the modified bacteria of claim 17.


SEQUENCE LISTING (1 sequence)

SEQ ID NO: 1 - DNA, 110 bp, Pseudomonas aeruginosa
  source 1..110: mol_type = other DNA
gggcaggataggtgaagtaggcccacccgcgagcgggtgttccttcttcactgtcccttattcgcacctggcggtgctcaacgggaatcctgctctgcgaggctggccgg